(19) United States
(12) Patent Application Publication
(10) Pub. No.: US 2024/0325601 A1
(43) Pub. Date: Oct. 3, 2024

(54) BONE GROWTH COMPOSITIONS AND ASSOCIATED TECHNIQUES

(71) Applicant: Warsaw Orthopedic, Inc., Warsaw, IN (US)

(72) Inventors: Benjamin T. Reves, Memphis, TN (US); Daniel A. Shimko, Germantown, TN (US); David R. Kaes, Toms River, NJ (US)

(21) Appl. No.: 18/622,029

(22) Filed: Mar. 29, 2024

Related U.S. Application Data
(60) Provisional application No. 63/493,585, filed on Mar. 31, 2023.
(60) Provisional application No. 63/510,961, filed on Jun. 29, 2023.

(51) Int. Cl. A61L 27/36; A61L 27/02

(52) U.S. Cl. CPC A61L 27/3608; A61L 27/025; A61L 2430/02

(57) ABSTRACT

A bone growth composition may include a cancellous portion and a noncancellous portion. The cancellous portion may be processed with an antimicrobial agent contact step and subsequent contact with a cryopreservative solution. In an embodiment, the cryopreservative solution is glycerol. In an embodiment, the bone growth composition includes a mix of cancellous chip formed from the cancellous portion and demineralized fibers formed from the noncancellous portion.

### CROSS-REFERENCE TO RELATED APPLICATION

This application claims priority and benefit of U.S. Patent Application No. 63/493,585, entitled “Bone Growth Compositions and Methods,” filed on Mar. 31, 2023 and U.S. Patent Application No. 63/510,961, entitled “Bone Growth Compositions and Associated Techniques,” filed on Jun. 29, 2023, both of which are incorporated herein by reference in their entirety.

### BACKGROUND

The present disclosure relates generally to bone growth compositions, such as cellular bone matrix compositions, and techniques associated with bone growth compositions.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In clinical use, a variety of conditions may warrant repair and/or replacement of an internal body part, such as bone. For example, to repair a bone fracture, an adhesive agent may be applied to adhere sections of the separated bone together. A bone filler material may be applied to a bone in a subject to replace degenerated tissue and/or to provide a supportive matrix to support or reinforce the bone and promote bone growth.

Since the early 1990s, carefully processed allograft bone from donated human tissues have been routinely transformed into various type of bone grafting materials with many derivations in shape, form, and purpose. The first standards for tissue banking published by the American Association of Tissue Banks (AATB) in 1984. Due to an increasing medical and market demand for orthopaedic implants, a selection of grafting options has been developed. Fresh frozen allograft bone (derived from either deceased donors or surgical discards) has been used to supplement the available autograft bone recovered from patients undergoing surgery and in need of bone grafting. Osteotech launched the first formulated demineralized bone matrix product for bone grafting called Grafton™ Gel in 1991.

Commercial products intended as an alternative to autologous grafts have included growth factors, or a combination of natural or synthetic scaffold materials. While the use of demineralized bone fibers (DBF) creates structural matrices and promotes bone growth, the use of DBF alone lacks the viability of fresh tissue. Certain techniques may use cell-containing bone materials. However, clinical use of cell bone matrices has been associated with significant issues, such as high cost, burdensome and nonoverlapping regulatory matters globally, storage and transport challenges, and may infer complications associated with immunological response.

Cellular matrix products manufactured in the United States are regulated by section 361 of the Public Health Service Act and Code of Federal Regulation (CFR) title 21 section 1271, which does not require Food and Drug Administration (FDA) premarket review and approval. As such, the products will be considered human cells, tissues, or cellular or tissue-based products (HCT/Ps) so long as the products satisfy the following criteria: minimal manipulation; homologous use only; systemic effect absence; the primary function not dependent on the metabolic activity of viable cells, unless the product is intended for autologous use or use by a first- or second-degree blood relative.

Certain existing products on the market are also failing in forming stable spinal fusion, for example, in posterolateral fusion. Efficacy and safety studies, large randomized clinical trials, and additional research are needed to solidify the role of allograft with viable cells, especially in current studies on animal models that show controversial data. Cellular autograft, cellular bone matrix (CBM)/viable bone matrix (VBM) s, also need better understanding as to which product best aligns with a specific indication and cost-effective solution.

For each CBM, however, several intrinsic biological characteristics, such as viable cell sources, the donor age at the time of graft harvest, or cell survival after transplantation, cause variations among different lots of the same product in terms of expected outcomes. Processing may also introduce variability in product; such variability may include cell type, cell amount, cell viability dependent on donor material, cell viability after thawing, bone tissue processing, subsequent formulation, cryoprotectant agents, cryopreservation or freezing, and storage.

Further issues in regards to maintaining viability of cells remain. Cryoprotectant agents utilized to maintain cell viability at extremely low temperatures (e.g., less than about-80° C.) and for long-term storage and transport, e.g., dimethyl sulfoxide (DMSO), have an intrinsic cytotoxicity that requires rapid removal from grafts before implant. In fact, cryoprotectants during thaw cause cell death, therefore requiring additional rinsing, decanting, and handling that may be detrimental to the viable cells. Use of other cryoagents, or storing at less extreme cold temperatures has caused shrinking of the cells and/or caused formation of intracellular ice (which causes the cell to burst upon thaw).

A need exists to develop a CBM/VBM product that maintains a cellular viability similar to the live cell count of the extracted bone particles, granules, or fibers prior to processing. A CBM product is needed that will address the issues as described above.

### SUMMARY

Certain embodiments are summarized below. These embodiments are not intended to limit the scope of the disclosure. Indeed, the present disclosure may encompass a variety of forms that may be similar to or different from the embodiments set forth below. The bone growth composition of the current application will address the issues discussed prior, and further include an improved capability for use, manipulation, preservation, thaw and post-thaw applications. Such improvements are described herein.

In one embodiment, a bone growth composition is provided. The composition comprises demineralized bone fibers generated from a donor bone tissue; cancellous chips generated from the donor bone tissue; and a cryopreservative solution comprising glycerol in Lactated Ringer's, wherein a glycerol content of the cryopreservative solution is equal to or less than 25% by volume.

In such an embodiment, various implementations may be provided, which may be implemented alone or in any suitable combination with one another. By way of example, in one implementation the cancellous chips are antimicrobial-treated with an antimicrobial agent. In an aspect of such an implementation the antimicrobial agent is not present or present in residual amounts of less than 1% by mass in the bone growth composition. In another implementation the demineralized bone fibers have an average length equal to or less than 5 mm and range in thickness between 0.05 to 0.5 mm. In another implementation the bone growth composition is enclosed in a container or a pouch. In another implementation the glycerol content of the cryopreservative solution is equal to or less than 10% by volume. In another implementation the cancellous chips and the fibers are present in about a 1:1 volume ratio. In an aspect of such an implementation there is less than 100% homogeneity of a cancellous portion of the donor bone tissue that is segmented and subsequently processed into the cancellous chips such that cancellous chips comprise at least 1% noncancellous material. In another aspect of such an implementation there is less than 100% homogeneity of a cortical or noncancellous portion of the donor bone tissue that is segmented and subsequently processed into the demineralized bone fibers, and wherein there is less than 100% homogeneity of the cortical portion such that the demineralized bone fibers comprise at least 1% noncortical material. In another implementation the bone growth composition comprises one or both of corticocancellous chips and cortical chips in addition to the cancellous chips.

In one embodiment a frozen bone growth composition product is provided. The product comprises a sealed flexible pouch enclosing a bone growth composition, the bone growth composition comprising: demineralized bone fibers generated from a donor bone tissue; cancellous chips generated from the donor bone tissue; a cryopreservative solution comprising glycerol in Lactated Ringer's, wherein a glycerol content of the cryopreservative solution is equal to or less than 20% by volume, wherein the demineralized bone fibers, the cancellous chips, and the cryopreservative solution are all frozen such that the bone growth composition is solid.

In such an embodiment, various implementations may be provided, which may be implemented alone or in any suitable combination with one another. By way of example, in one implementation the cancellous chips and the fibers are present in about a 1:1 volume ratio. In another implementation the bone growth composition is frozen at a temperature from about-65° C. to about −80° C. In another implementation the bone growth composition is frozen at a temperature less than about −65° C. and wherein a viability of cells of the bone growth composition after thawing is more than 75-80%. In another implementation a total volume of the bone growth composition is 15 cubic centimeters or less. In another implementation the glycerol content of the cryopreservative solution is equal to or less than 10% by volume.

In one embodiment a thawed bone growth composition product is provided. The product comprises a sealed flexible pouch enclosing a bone growth composition comprising: demineralized bone fibers generated from a donor bone tissue; cancellous chips generated from the donor bone tissue; and a cryopreservative solution comprising glycerol in Lactated Ringer's, wherein a glycerol content of the cryopreservative solution is equal to or less than 20% by volume, wherein the demineralized bone fibers, the cancellous chips, and the cryopreservative solution are all thawed such that the bone growth composition is flowable within the sealed flexible pouch.

In such an embodiment, various implementations may be provided, which may be implemented alone or in any suitable combination with one another. By way of example, in one implementation a total decantable volume of the cryopreservative solution after thawing is between about 0 mL to about 1 mL. In another implementation the sealed flexible pouch comprises a removable release liner covering a perforated wall. In another implementation the sealed flexible pouch is stored at 25° C. or higher after thawing. In another implementation the sealed flexible pouch has a first dimension longer than a second dimension to form an elongated shape.

### BRIEF DESCRIPTION OF THE DRAWINGS

Advantages of the disclosed techniques may become apparent upon reading the following detailed description and upon reference to the drawings in which:

FIG. 1 is a flow diagram of a processing method to produce a cellular bone matrix from donor bone tissue, in accordance with an embodiment of the present disclosure;

FIG. 2 shows an example bone region including sources for cancellous, cortical, and corticocancellous chips, in accordance with an embodiment of the present disclosure;

FIG. 3A shows an example bone region including sources for cancellous, cortical, and corticocancellous chips, in accordance with an embodiment of the present disclosure;

FIG. 3B shows an example bone region including sources for cancellous, cortical, and corticocancellous chips, in accordance with an embodiment of the present disclosure;

FIG. 4 is a flow diagram of a fiber processing method that may be used in conjunction with FIG. 1, in accordance with an embodiment of the present disclosure;

FIG. 5 shows example fibers, in accordance with an embodiment of the present disclosure;

FIG. 6 shows SEM images of demineralized fiber samples that were freeze-dried, air-dried, or sCO2-dried, in accordance with an embodiment of the present disclosure;

FIG. 7 is a flow diagram of a two-step chip washing method, in accordance with an embodiment of the present disclosure;

FIG. 8 is a flow diagram of a two-step chip washing method, in accordance with an embodiment of the present disclosure;

FIG. 9 shows effects of the two-step chip washing method on a tissue sample relative to a control;

FIG. 10 is a flow diagram of chip washing, in accordance with an embodiment of the present disclosure;

FIG. 11 shows a comparison of cell viability for different washing techniques;

FIG. 12 shows a comparison of cell viability for different washing techniques;

FIG. 13 shows a comparison of cell viability for different cryopreservative solutions;

FIG. 14 shows a comparison of cell viability for different cryopreservative solutions;

FIG. 15 is a flow diagram of a method of contacting chips with a cryopreservative solution for freezing, in accordance with an embodiment of the present disclosure;

FIG. 16 shows example components of a bone growth composition, in accordance with an embodiment of the present disclosure;

FIG. 17 shows an example bone growth composition packaged in a pouch and removed from an outer packaging, in accordance with an embodiment of the present disclosure;

FIG. 18 shows an example bone growth composition packaged in a pouch, in accordance with an embodiment of the present disclosure;

FIG. 19 shows an example bone growth composition packaged in a pouch and in a water bath for thawing, in accordance with an embodiment of the present disclosure;

FIG. 20 shows an example bone growth composition packaged in a pouch and being opened, in accordance with an embodiment of the present disclosure;

FIG. 21 shows an example of different conditions for a bone growth composition packaged in a pouch, in accordance with an embodiment of the present disclosure;

FIG. 22 shows an example bone growth composition packaging system including a release liner that is removed to permit draining, in accordance with an embodiment of the present disclosure;

FIG. 23 is a flow diagram of assessing cell viability, in accordance with an embodiment of the present disclosure;

FIG. 24 shows a comparison of cell viability for different freezing techniques;

FIG. 25 shows a comparison of cell viability for different antimicrobial agents;

FIG. 26 shows a comparison of cell viability for different antimicrobial agents with respect to soak temperature;

FIG. 27 shows a comparison of cell viability for different antimicrobial agent concentrations;

FIG. 28 shows a comparison of cell viability for different antimicrobial agent rinse conditions;

FIG. 29 shows a comparison of post-thaw cell viabilities;

FIG. 30 shows a comparison of cell stability for different storage time conditions; and

FIG. 31 shows histology images containing a small amount of potentially immunogenic donor-derived blood cells.

### DETAILED DESCRIPTION OF SPECIFIC EMBODIMENTS

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The methods discussed herein include various steps represented by blocks in flow diagrams. It should be noted that at least some steps may be performed as an automated procedure by one or more components of a system. Although the flow diagrams may illustrate the steps in a certain sequence, it should be understood that the steps may be performed in any suitable order and certain steps may be carried out simultaneously, where appropriate. Additionally, steps may be added to or omitted from of the methods.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements. One or more specific embodiments of the present embodiments described herein will be described below. In an effort to provide a concise description of these embodiments, all features of an actual implementation may not be described in the specification. It should be noted that in the development of any such actual implementation, as in any engineering or design project, numerous implementation-specific decisions must be made to achieve the developers' specific goals, such as compliance with system-related and business-related constraints, which may vary from one implementation to another. Moreover, it should be noted that such a development effort might be complex and time consuming, but would nevertheless be a routine undertaking of design, fabrication, and manufacture for those of ordinary skill having the benefit of this disclosure.

The disclosed bone graft compositions include demineralized allograft bone fibers from cortical bone, and cancellous and corticocancellous bone chips, and glycerol. The chips, being fully mineralized, retain their inherent radiopacity and enhance detection of the implant when using x-ray. The mineralized chips and demineralized fibers also impart desirable handling characteristics to the graft.

New bone formation in patients undergoing bone graft requires three elements; local signaling to drive new bone formation, a scaffolding material to template the shape of the future bone, and bone forming cells like osteoblasts and/or bone progenitor cells. The demineralized bone fibers expose local anabolic growth factors inherent in the tissue matrix. The demineralized fibers and cancellous bone chips also offer a scaffold material. In contrast to products in which the cells were provided by the recipient, the disclosed products provide allograft bone-forming cells.

The disclosed embodiments relate to bone growth compositions, such as bone matrix compositions. The bone matrix compositions may include cellular bone matrix compositions and techniques, also referred to as cellular bone matrices (CBMs) or viable bone matrices (VBMs). As provided herein, cellular bone matrixes provide a platform for new bone formation in a subject. A mix of bone elements and bone elements that have live bone cells provides an osteoconductive scaffold and viable cells with osteogenic potential. Specifically, the CBMs provide various components for new formation, namely, an osteoconductive scaffold, extracellular growth factors for cell proliferation and differentiation, and viable cells with osteogenic potential. The growth factors and proteins in the demineralized fibers provide an active signal (i.e., osteoinductivity) for bone healing. Both the chips and fibers act as scaffolds for bone regeneration (i.e., osteoconductivity). Cellular bone matrices may be used for bone grafts, e.g., allogenic bone grafts. Cellular bone matrices include live bone cells, such as live mesenchymal stem cells (MSCs).

The CBM product is an autograft extender having osteoconductive, osteoinductive and osteogenic characteristics. The cryopreservative solution, e.g., a glycerol and lactated Ringer's solution, has been optimized to retain cellular viability. Also provided herein are effective manufacturing methods to optimize the manufacturing of this formulation in a way that maintains high cell viability, enhances microbiological safety, and enables high osteoinductive performance. These are described in more detail herein.

By design, the CBM is stored frozen using agents to maintain the viability of cells contained within the tissue. The tissue is prepared and packaged in a way that makes it ready-to-use following a short duration thawing process. The CBM can be utilized alone or mixed with autograft to enhance osteogenic characteristics and encourage bone fusion. The CBM may be moldable for insertion into a spinal implant cage or conforming to the contours of bone or other spinal region. Where the CBM is utilized for packing in a cage implant, the product may be compacted and malleable to conform to the designated space. In some instances, the composition can be dried by squeezing to alter its handling characteristics to the preference of the user. The composition alternatively be easily hydrated with fluid, water, blood, marrow, or otherwise to alter handling and increase malleability. Further hydration can enhance the compositions flowability for use with a graft insertion gun or funnel and enable it to be easily pressed through long cannulas less than about 8 mm in diameter. Once compacted or injected at the site for spinal repair, the graft is stable and unlikely to migrate due to the inherent cohesivity of the formulation. In some cases, the formulation is resistant to gentle irrigation.

Regarding appearance, the CBM has a fibrous appearance with particulate heterogeneity. In comparison with prior technology utilizing terminal sterilization, the embodiments herein comprise one or more steps of aseptic processing, variable according to batched donor supply and/or standardized for consistency across product line.

The improved thawing characteristics of the CBM include flexible packaging to permit an expanded surface area exposed to thawing temperatures and minimization of air pockets that inhibit thawing.

The bone repair donor materials disclosed herein may be autologous, allogenic, or xenogenic. For example, an autograft, as used herein, may refer to bone and/or tissue that is extracted from the intended recipient of an implant. An allograft refers to bone and/or tissue that is from a donor that is different than the intended recipient. A xenograft refers to bone and/or tissue that is from a donor that is a different species than the intended recipient. The disclosed compositions and techniques may be used in the context of autologous, allogenic, or xenogenic compositions or mixtures thereof. Further, the disclosed products may be formed from one or more donors, e.g., a single product may be formed from a single donor or from donor materials from multiple donors. For example, in certain cases, an allogenic material may be mixed with an autograft material to form a final product that is provided to the patient. The donor bone tissue may be cadaveric or from a living donor. In an embodiment, the bone growth composition uses tissue originating from a recently deceased human and is processed within a sufficiently short timeframe to enable retention of cellular components. The donor bone tissue may be a bone end of a long bone, from bone locations proximal to joints, or within the interior of vertebrae. By way of example, the donor bone tissue may be a distal end radius, proximal humerus, or proximal femur.

In embodiments, tissue recovered for use in the CBM product will be from donors screened, recovered, and tested in compliance with current AATB and FDA guidance documents. In an example, the source bone will originate anatomically from the extremities and from portions of the pelvis. In an embodiment, bone originating from the spine, ribs, cranial region, hand, or foot anatomy is not used. The disclosed embodiments may include testing and exclusion of any donor that tests positive for HTLV I/II. Donors testing positive for certain markers, such as Cytomegalovirus IgG and IgM antibodies by ELISA, may also be excluded. Donor tissue which tests positive for Streptococcus pyogenes, Clostridium sp, or Mycobacterium tuberculosis organisms (by PCR and/or culture) may also be excluded.

In an embodiment, the donor tissue is in compliance with AATB bulletin 22-2. This document makes additional recommendations for tissue banks to consider to limit the risk of Mycobacterium tuberculosis transmission. The CBM products may exclude tissue from donors with a history of dialysis outside of an ICU setting or donor with a history of long-term steroid use prior to death.

Donor tissue is recovered within the time period defined by AATB guidelines. Following recovery, tissue is stored and transported to on wet ice or other appropriate conditions per AATB guidelines. In an embodiment, donor tissue may not be frozen prior to processing into the bone composition product. In an embodiment, donor tissue must complete its processing into the finished product within 96-120 hours of donor time of death or, in an embodiment, within 96 hours of donor time of death.

For clarification, viable cells within CBMs are cell populations capable of promoting synthesis of new bone, such as multipotent adult progenitor cells (MAPC), mesenchymal stem cells (MSCs), osteoprogenitor cells (OPCs), osteocytes, and/or osteoblasts (OBs). MAPCs and MSCs are both non-hematopoietic cells found in the bone marrow stroma; these cells retain the ability to self-replicate and differentiate into a specific phonotype by intrinsic and local environmental cues (e.g., spatial organization, mechanical forces, growth factors). Many other cells common to bone tissue may also be present in CBMs. Those could include fibroblasts, adipocytes, vascular endothelial cells, hematopoietic cells, lymphocytes, and other cells found commonly in bone.

In certain embodiments, techniques for preparing, manufacturing, and/or storing cellular bone matrix compositions are provided. In certain embodiments of the invention the disclosed techniques permit improved biological activity (e.g., osteoinductivity and/or osteoconductivity) of cellular bone matrices at one or more preparation stages. The cellular allografts, CBM/VBMs, are obtained by processing to largely remove immune-responsive signals generated by bone marrow components. Such components could include hematopoietic cells, retaining bone-forming cells within the bone matrix.

Embodiments of the disclosed techniques relate to cellular bone matrix processing methods that include an antimicrobial agent contact step. Use of an antimicrobial agent during processing may improve clinical results and end product characteristics by reducing potential for spoilage and improving shelf life of the cellular bone matrix. This contact step may also be used to reduce or eliminate microbiological contamination originating from the starting tissue or manufacturing process itself. However, the antimicrobial agent contact during processing may reduce cell viability for active cells of the chips. Certain antimicrobial contact conditions as disclosed herein may be associated with relatively higher cell viability. In addition, the disclosed techniques permit inclusion of an antimicrobial contact step in cancellous chip processing by maintaining or improving cell viability at other steps in chip processing, such that cell loss or reduced viability as a result of the antimicrobial contact step is compensated for by improved cell viability at other stages. These high viabilities in the manufacturing work flow result in a finished product with high cellular viability even while including aggressive antimicrobial steps to minimize the potential for bacterial contamination in the finished product while maintaining high performance.

In particular, cryopreservatives that permit storage of the cellular bone matrix products may nonetheless have harmful effects on cell viability. Embodiments of the disclosed techniques include cold preservation and cryopreservation in cryopreservative formulations and conditions with associated improved cell viability characteristics. The disclosed techniques also include cellular bone matric processing with wash or rinsing step conditions with unexpected cell viability benefits relative to conventional processing steps. In embodiments, the disclosed techniques include contacting a cancellous portion with glycerol combined with Lactated Ringer's at glycerol concentrations that, for example, provide enhanced cell viability at one or more freezing temperature conditions. In certain cases, the disclosed product can be maintained for two or more months (e.g., six months or more) at −70 to −80° C. storage conditions.

Although popular among surgeons, many cellular bone matrices have complicated thawing and preparation steps. For example, thawing in a jar or rigid container can take up to 30 minutes. In contrast, in an embodiment, the disclosed cellular bone matrix compositions are frozen in flexible bags, flexible pouches, vessels, or receptacles such as a pouch. The CBM is frozen in pouches as the primary packaging which permits much faster thawing (<15 minutes). This facilitates access and quick access in a surgical setting. In one aspect, the malleable pouch allows for external manipulation of the CBM as it thaws. In addition, many cellular bone matrices require that the cryopreservative solution be poured/decanted off after thawing because of dimethyl sulfoxide (DMSO) toxicity. Sometimes, the tissue has to be rinsed with saline. In contrast, the disclosed cellular bone matrix compositions do not require decanting or washing to maintain high cell viability after thawing. If, however, the user decants off any volume of liquid or storage solution, the pouch can be pinched off at the end to facilitate decanting. As well, a modified end portion of the pouch may incorporate a perforated sheet integrated with the sidewalls of the pouch to contain the CBM while allowing the extra volume of liquid to be easily extracted or poured off. As well, CBM packaged within individual cannulas internal to the pouch will be capable of being frozen and thawed from a freezing state in reduced times compared to that of a jar. These features enhance end user workflow relative to products that involve more complex handling.

A cellular bone matrix composition may be formed from a cancellous portion of a bone (e.g., cancellous chips) and a noncancellous, e.g., cortical or compact, portion of the bone (e.g., cortical fibers). One embodiment of the noncancellous portion of bone includes cortical bone particles, pieces, segments, and/or fibers. When harvesting or grating fibers from the donor tissue, the fibers are produced in a ribbon-like configuration, the fibers of which are irregular in shape with expansive surface areas. For exemplary purposes, and not limitation, the ribbon-like structures maintain the ribbon-like configuration that is a columnar-like sheet that wraps around itself. The ribbon-like configuration has increased surface area via the curled portion and, in embodiments, surface nanostructures. In some embodiments, nanostructures are imparted to bone matrix compositions wherein the nanofibrous properties on the surface may be compromised. In other embodiments, the bone is processed in a manner so as to expose and/or maintain the nanofibrous structure of the bone. The nanostructures may be as disclosed in U.S. Pat. No. 10,220,115, the disclosure of which is hereby incorporated by reference in its entirety herein.

The particulates or fibers harvested range in size with variations from millimeters to centimeters, as described herein. At the end stage of milling, the particulates and fibers may range in size to form a non-homogenous fiber mix. In an embodiment, the fibers may range in micron sizes from about 50 microns to 5 cm or 10 cm. In an embodiment, the noncancellous portion contains less than 10% soft tissue. Both the chips and fibers, when provided as a cellular bone matrix, act as a scaffold for bone formation to a subject in need of bone repair. The cancellous portion, which contains viable bone cells, is processed separately from the noncancellous portion during cellular bone matrix manufacturing to retain the viability of the bone cells during processing. That is, processing steps applicable to the cortical fibers may be unsuitably harsh for the cancellous chips. However, certain processing steps to clean, disinfect, or cryopreserve the cancellous portion may nonetheless reduce bone cell viability and osteoinductivity. In an embodiment, the disclosed techniques permit processing of the cancellous portion to clean, disinfect, and/or cryopreserve while maintaining adequate bone cell viability. Thus, the cellular bone matrix end product produced by the disclosed techniques has desirable clinical characteristics.

The mix of chips and demineralized fibers in cellular bone matrix compositions as provided herein provide excellent handling. The fibers yield a cohesive putty-like implant, and the mineralized chips provide some grittiness. Internal testing has shown that the disclosed cellular bone matrix compositions are osteoinductive in the gold standard athymic rat muscle pouch model.

In practice, such a formulation may be provided as a human cryopreserved viable cortical cancellous bone matrix and demineralized bone fibers. The allografts may be prepared using aseptic surgical techniques and may be further processed and packaged under aseptic conditions. The viable cortical cancellous bone matrix component may be prepared from milled bone cleaned using purified water and lactated Ringer's (or other suitable agents) and may be treated with antibiotics (gentamicin, vancomycin) and antimycotic (amphotericin B) agents as discussed herein. The demineralized bone fibers may be prepared from milled bone cleaned using purified water or other suitable agents. These fibers may be demineralized to yield a demineralized bone matrix with a suitable calcium content level, such as a level that meets current American Association of Tissue Banks (AATB) standards. As discussed herein, the formulated and packaged tissue may be soaked and stored in a biocompatible cryoprotectant solution containing glycerol and lactated Ringer's solution to help retain cell viability of the cortical cancellous bone matrix during frozen storage. As further discussed herein, the presently contemplated cellular bone matrix includes demineralized bone allograft fibers that are osteoconductive as well as osteoinductive.

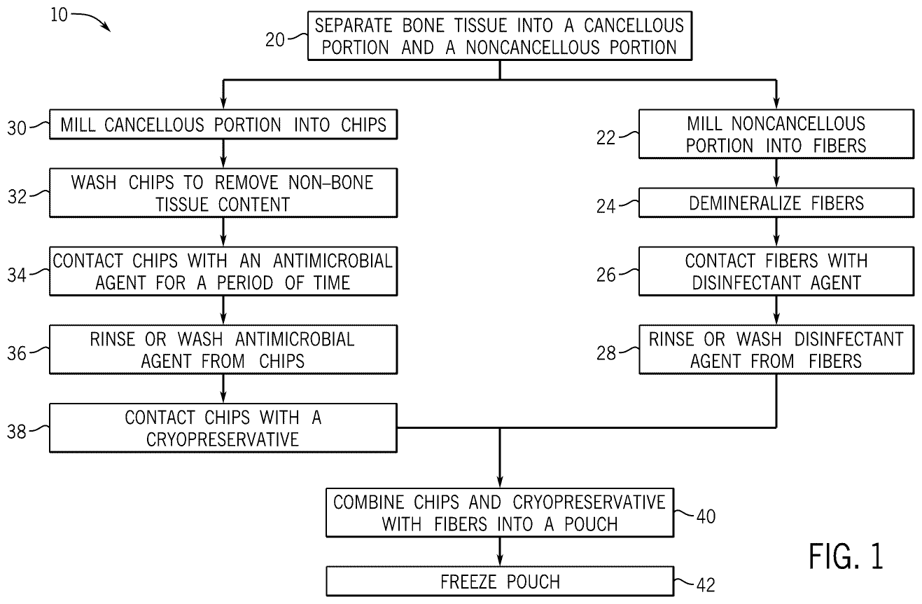

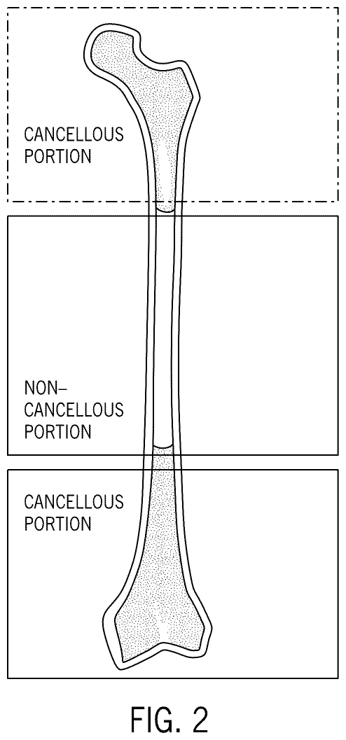

With the preceding in mind, FIG. 1 depicts a processing method 10 to produce a cellular bone matrix from donor bone tissue. At step 20, the donor bone tissue is separated into a cancellous portion and a noncancellous portion, as shown by way of example in FIG. 2. In an embodiment, the donor bone tissue exterior is cleaned. Donor bone treatment may also include a debridement step to remove non-bone tissue content prior to separation. In one aspect, a high-pressure water cleaning process is used to clean the bone. In embodiments, the donor tissue is provided without having been previously frozen to maintain cell viability. Thus, the disclosed bone growth compositions, after freezing, may be considered fresh-frozen. The separation into the cancellous portion and the noncancellous portion may be via cutting or sawing techniques to remove cancellous tissue from the distal and proximal ends of a bone shaft. The cancellous portion and the noncancellous portion may be cut or segmented during or after separation into blocks that are suitably-sized for downstream steps of the process 10.

As is often the case, noncancellous bone is attached to or encasing or surrounding the cancellous bone elements of the tissue being segmented from the native anatomy. Thus, as provided herein, the cancellous portion may also include noncancellous elements in some nonzero percentage that are incorporated and retained within the subsequent chip form. Further, as provided herein, the noncancellous portion and the cancellous portion may not be achieved with 100% homogeneity. In fact, this imprecise segmentation is desirable as it minimizes donor material loss by eliminating unnecessary cutting and trimming of the tissue during segmentation. It should be understood that the cancellous and noncancellous portions of bone as described herein are predominately cancellous bone or noncancellous bone, but comprise some elements of both. In an example, the cancellous portion and the noncancellous portion may be cut into blocks that are between 3-10 cm in one dimension. Processing may be modified here to cut at a desired size. As well, the tools utilized for creating the particulates or fibers may determine the portion sizes created.

Turning to the processing of the noncancellous portion as discussed in FIG. 1, the noncancellous portion, at step 22, is milled or otherwise processed (e.g., cut, grated, shredded) into fibers. The fibers may be any suitable size or shape. In an embodiment, the fibers have a length dimension that is at least twice a width and height dimension. In an embodiment, the fibers have an aspect ratio of at least 50 (length):1 (height and/or width), and an average length greater than 0.5 cm. In an embodiment, the width and height dimension of the fiber have about a 1:1 aspect ratio while the length dimension is elongated. In an embodiment, a fiber length dimension is at least 5, 100, or 1000 times a width and height dimension. Example ranges of a length dimension relative to other dimensions are between 5-50 times, 5-100 times, or 5-1000 times. In an embodiment, the fibers may be uniform or nonuniform, e.g., may have a uniform or nonuniform length, width, and or surface area. In an embodiment, the fibers may be curled or ribbon-like. In an embodiment, the fibers are less than 10 or 5 mm in any dimension. In some embodiments, the bone fibers have a diameter from about 100 μm to about 2 mm. In some embodiments, the bone fibers have a length from about 0.5 mm to about 50 mm. In some embodiments, the bone fibers have an average length from about 0.5 cm to about 10 cm. In an embodiment, the average bone fiber thickness is between 0.05 to 0.5 mm.

In one embodiment, the bone fibers are milled using an osteobiologic milling machine as disclosed in U.S. Pat. No. 9,004,384, the disclosure of which is hereby incorporated by reference in its entirety for all purposes. The osteobiologic milling machine may be computer-controlled, with instructions that are programmed, stored, and/or executed by a programmable logic controller. The instruction may control milling parameters such as fiber length, thickness, and/or surface area. In an embodiment, the output of the osteobiologic milling machine may include material that is outside of tolerance. Thus, in an embodiment, the output may undergo a sorting or sieving step to remove bone components that are too long, too short, too thin, or too thick.

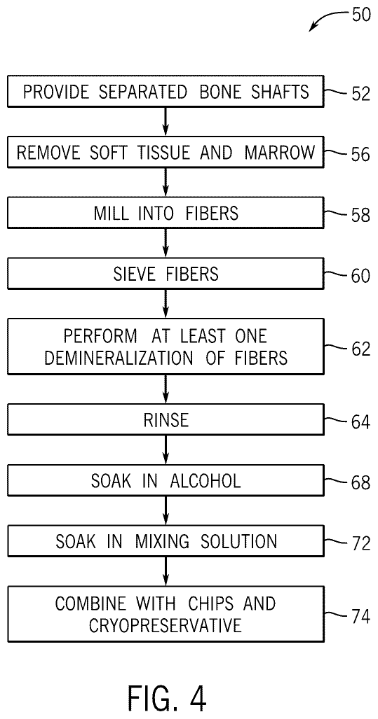

At step 24, all or some of the fibers are demineralized, which may refer to a process of removing inorganic content, such as minerals, from cortical fibers. The fiber end product after demineralization may have at least 50% or at least 90% or at least 99% of minerals (e.g., calcium) removed. In an embodiment, the fibers may be demineralized such that the fiber end product after demineralization has less than 1% residual calcium. Demineralization may occur via an acid soak (e.g., HCl, 0.6N HCl). At step 26, the demineralized fibers are disinfected with an alcohol solution (70% alcohol in water by volume) and at step 28 rinsed/transferred to a solution, such as Lactated Ringer's, to soak before being combined with the processed cancellous portion at step 40. An embodiment of cortical fiber processing is discussed in more detail in FIG. 4.

Demineralized bone matrix (DBM) has been shown to exhibit the ability to induce and/or conduct the formation of bone. Demineralization improves flexibility and handling characteristics. Also, calcium/mineral phase of bone can cover the collagen phase of bone. Demineralization exposes the bone growth factors to facilitate bone growth. It is therefore desirable to implant and maintain demineralized bone matrix at a site which bone growth is desired. Bone fiber based-demineralized bone matrices for implantation exhibit improvements in mechanical properties, including cohesiveness, fiber length, fiber diameter or width, fiber aspect ratio, or a combination of multiple variables.

Turning to the processing of the cancellous portion, at step 30, the cancellous portion removed from the bone tissue is milled into chips. As disclosed, the cancellous portion may refer to a bone tissue portion that is majority or predominantly cancellous tissue (e.g., greater than 50%, greater than 75%, or greater than 90% by volume) but that nonetheless includes other elements, such as some encasing cortical tissue as well as marrow elements, blood, fat that remain before cleaning. Indeed, a benefit of the disclosed technique is that milling the cancellous bone with the marrow elements present protects the cells within the tissue to improve viability and/or cell retention at this stage.

While fibers may be characterized by an elongated form, a chip in certain embodiments may have a planar form having a width dimension that is greater than a width of a fiber and a length dimension that is less than a fiber length while having a similar height dimension. In an embodiment, a chip aspect ratio is at least 50:1, and the chip an average length greater than 0.1 cm (e.g., 0.5 mm-2 mm). In an embodiment, the chips may be uniform or nonuniform. In an embodiment, the chips are less than 5 mm in any dimension.

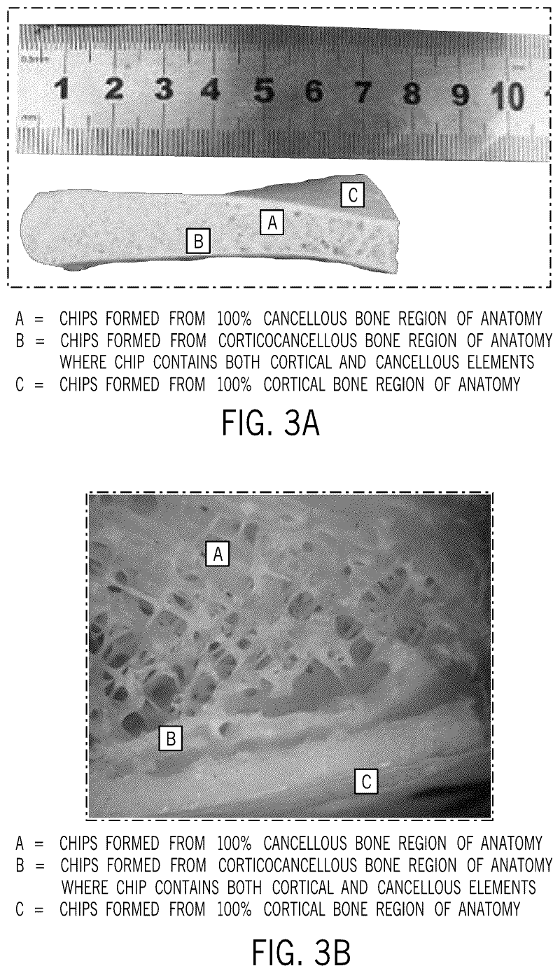

FIGS. 3A-B show an example cancellous portion of a bone tissue (see FIG. 2), e.g., an iliac wedge, that is processed to form chips and that is predominantly cancellous tissue but that includes example cancellous (A), corticocancellous (B), and cortical (C) bone regions. The cancellous, cortical, and corticocancellous bone regions are respective sources for cancellous, cortical, and corticocancellous chips. Accordingly, the overall composition of chips as used herein may be cancellous chips or some combination (i.e., a mixture) of cancellous chips, corticocancellous chips, and cortical chips. As discussed, in certain embodiments the population of chips is predominantly cancellous (e.g., greater than 50%, greater than 75%, greater than 95%) by percentage. However, a certain percentage of chips include cortical or noncancellous tissue. Further, an individual chip of the chips may be comprised of 100% cancellous tissue, 100% cortical tissue, or may be a corticocancellous chip generated from a corticocancellous region, e.g., a heterogenous mixed chip that includes both cancellous tissue and cortical tissue present on a single chip. In one example, cancellous chips may include a population of chips are 50-99% cancellous with the remaining chips being one or more of noncancellous or mixed cancellous/noncancellous. In one example, the chips are about 80-90% cancellous and about 10-20% mixed cancellous/noncancellous. In one example, the chips are about 90-99% cancellous and about 1-9% mixed cancellous/noncancellous. The cancellous chips as provided herein may refer to a set or population of chips that is at least 50% cancellous. Cortical chips may be less porous and more dense than cancellous chips. Thus, while the chips may be sized and shaped similarly, their structural qualities may be different. In an embodiment, cancellous chips have at least 1% noncancellous materials. Further, demineralized bone fibers formed from the cortical portion may also have a heterogenous composition that includes at least 1% noncortical materials.

The chips are washed at step 32 to begin removal of non-bone tissue elements and contacted with an antimicrobial agent at step 34. The chips may be soaked in the antimicrobial agent for a period of time, e.g., a predetermined period of time. In an embodiment, the antimicrobial soak is conducted in a temperature range of 25-37° C. and for at least 1 hr or in a range of 3 hrs-12 hrs. In an embodiment, the antimicrobial soak is conducted at 35-37° C. or 37° C. and for at least 5 hours but no more than 10 hours. The antimicrobial solution is selected based on characteristics of the antimicrobial agent and at a concentration sufficiently high to kill undesirable microorganisms. In an embodiment, the antimicrobial agent is mixed with water (e.g., filtered water). In an embodiment, the antimicrobial agent is mixed with an isotonic solution (e.g., lactated Ringer's solution, saline, etc.). In an embodiment, the soak is conducted with agitation and/or stirring.

After the antimicrobial contact, the chips are washed to remove the antimicrobial agent (or to leave only residual amounts of the antimicrobial agent) using one or more washes at step 36. The wash step can use Lactated Ringer's (e.g., Lactated Ringer's solution or Ringer's lactate) to facilitate transition to the cryopreservative contact at step 38. In certain embodiments, the wash or rinse is conducted multiple times, e.g., three times, with removal of rinse solution between the rinses. In certain embodiments, the wash or rinse is timed (e.g., 5-15 minutes), and includes agitation (e.g., light stirring, etc.). After the antimicrobial contact, the chips are antimicrobial-treated chips. At step 38, the antimicrobial-treated chips are soaked in a cryopreservative solution. In an embodiment, the cryopreservative solution is 10-25% glycerol by volume and 75-90% mixing solution. In an embodiment, the cryopreservative solution is 15% glycerol by volume, 85% mixing solution. In an embodiment, the mixing solution is Lactated Ringer's. In an embodiment, the cryopreservative solution is 20% glycerol (e.g., has a glycerol content of 20% by volume), 80% Lactated Ringer's. In an embodiment, the chips are soaked in the cryopreservative solution for a period of time, e.g., a predetermined period of time. In an embodiment, the chips are soaked in the cryopreservative solution for at least 30 minutes, at least 60 minutes, at least 120 minutes, or at least 180 minutes. In an embodiment, the chips are soaked in the cryopreservative solution for 30 minutes to 240 minutes. In an embodiment, the chips are soaked in the cryopreservative solution for 60 minutes to 180 minutes. The cryopreservative solution can be provided in sufficient volume to cover the chips. In certain embodiments, after completion of the soak, at least some of the cryopreservative solution can be decanted or removed in preparation for freezing.

In an embodiment the Lactated Ringer's as provided herein may be Lactated Ringer's (e.g., Baxter Lactated Ringer's from Baxter, B. Braun Lactated Ringer's from B. Braun Medical Inc.) having the following composition: each 100 mL of Lactated Ringer's contains: Sodium chloride 600 mg; sodium lactate, anhydrous 310 mg; potassium chloride 30 mg; calcium chloride, dihydrate 20 mg. The pH is 6.6 (6.0-7.5). One liter has an ionic concentration of 130 mEq sodium, 4 mEq potassium, 2.7 mEq calcium, 109 mEq chloride and 28 mEq lactate. The osmolarity is 525 mOsmol/L (calc).

In certain embodiments, the cryopreservative is glycerol, and the cryopreservative solution does not include DMSO. In an embodiment, cryopreservative is glycerol, and the cryopreservative solution does not include any additional cryopreservative agents. This cryosolution in combination with the viable bone cells and/or demineralized fibers may be referred to in embodiments as a cryo-ready product or bone growth composition.

At step 40, the chips and fibers are combined for storage. As discussed above, the chips are processed prior to freezing and complete processing wetted with cryopreservative solution. The fibers are processed and may complete processing with a soak or wash in the mixing solution without any cryopreservative, e.g., in a Lactated Ringer's solution. Thus, mixing of the chips and fibers dilutes the existing cryopreservative present with the chips. The amount of dilution depends on 1) the volume of Lactated Ringer's solution carried together with the fibers relative to the volume of cryopreservative solution carried together with the chips at combination and 2) the ratio of chips to fibers.

In an embodiment, the ratio of chips to fibers is 1:1. In some embodiments, the chips to fibers ratio is about 90:10, 80:20, 75:25, 70:30, 60:40, 50:50, 40:60, 30:70, 25:75, 20:80 and/or 10:90. In some embodiments, the chips to fibers ratio is in a range between 70:30 and 30:70, between 60:40 and 40:60, between 55:45 and 45:55, or between 51:49 and 49:51. The ratio can be assessed as a volume:volume or a weight:weight ratio.

After combination, the combined chips and fibers that form the cryo-ready product are sealed in an appropriate freezing container, e.g., a pouch, and exposed to a chilled environment, a cold environment, or a freezing environment at step 42. In embodiments, the cold environment is a refrigerator temperature that is 5° C. or less and/or between 0° C. and 5° C. In embodiments, the freezing environment is a cryogenic environment. In an embodiment, the freezing environment is 0° C. or less. In an embodiment, the freezing environment is 0° C. to −200° C. In an embodiment, the freezing environment is 0° C. to −20° C. In an embodiment, the freezing environment is −15° C. to −20° C. In an embodiment, the freezing environment is −15° C. to −100° C. In an embodiment, the freezing environment is −15° C. to −80° C. In an embodiment, the freezing environment is −40° C. to −80° C. In an embodiment, the freezing environment is −65° C. to −80° C. In an embodiment, the freezing environment is −70° C.+/−15° C. In an embodiment, the freezing environment is −80° C.+/−15° C.

In certain embodiment, the disclosed cryo-ready product provides improved stability and viability even if certain steps in the freezing, storage, transport, and/or thawing process are not performed according to recommended guidelines. Further, the disclosed cryo-ready product, when frozen or chilled, may maintain viability even during conditions in which enzymatic or other chemical processes of the viable cells are still active at a lower level based on the storage temperature. In an embodiment, the disclosed cryo-ready product may be stored at temperatures at which certain cell processes, such as enzymatic reactions, are not completely shut down.

FIG. 4 is an example fiber processing method 50 that may be used in conjunction with FIG. 1. At step 52, bone shafts separated from bone ends are provided, and soft tissue and marrow is removed at step 56. At step 58, bone shafts, which may be provided as segments (e.g., 50-150 mm long segments) are milled into fibers. The milling may be performed using a cartridge mill. The fibers are sieved and shard material can be removed during the process at step 60. Post-milling, the fibers undergo at least one and, in embodiments at least two demineralizations at step 62. The demineralization may be an acid soak, such as a 0.6 N HCl soak. In an embodiment, the first demineralization is a 15-150 minute acid soak with occasional stirring. In an embodiment, the second demineralization is a 45-260 minute acid soak with occasional stirring. During acid soaks, pH may be monitored with a target pH of less than 1. Soak time or other conditions can be adjusted to achieve the target pH.

The fibers are rinsed in water (e.g., filtered water) at step 64. In an embodiment, the rinse is conducted at least two or three times (e.g., ten-minute soaks with soak solution replacement), and the fibers are subsequently soaked in alcohol at step 68. The alcohol soak may be in 70% ethanol (e.g., 20-200 minutes). The alcohol soak is followed by a soak in the mixing solution used for the cryopreservative, e.g., Lactated Ringer's at step 72 until the measured pH is at least 3 (e.g., 5-800 minutes). The demineralized fibers are then combined with chips and cryopreservative at step 74. FIG. 5 shows example fibers.

In some embodiments, the bone fibers have an average length to average thickness ratio or aspect ratio of the fibers from about 50:1 to about 1000:1. In overall appearance the bone fibers can be in the form of ribbons, threads, narrow strips, and/or thin sheets. The elongated bone fibers can be substantially linear in appearance or they can be coiled to resemble springs. In some embodiments, the bone fibers have linear portions and coiled portions. In some embodiments, the bone fibers are of irregular shapes including, for example, linear, serpentine and/or curved shapes. In some embodiments, the fibers can be curled at the edges to have a substantially hemicircular cross-sections. In some embodiments, the fibers may be entirely or partially helical, circumvoluted or in the shape of a corkscrew. The elongated bone fibers can be demineralized however some of the original mineral content may be retained when desirable for a particular embodiment. The bone graft fiber may further comprise mineralized bone material.

The bone fibers may be elongated and curled to increase the surface area of the strips. The curled fibers may include frayed portions along the edges to facilitate interactions with other bone fibers. In some embodiments, the curled fibers are milled to have hooked portions along the edges of the fibers configured to engage with other fibers. The hooked portions may engage other hooked portions, frayed portions, straightened portions or curled portions of other fibers. The hooked and frayed portions and the curled shape of the fibers provide for entanglement between fibers such that the fibers may form a coherent mass without the need for a carrier or binding agent, as generally disclosed in U.S. Patent Publication No. US20210402060A1, the disclosure of which is incorporated by reference in its entirety herein. The fibers may include nanofibers of a submicron level or any fibers having at least one side or dimension at or below 1000 nanometers. In specific embodiments, the fibers may have at least one side or dimension at or below 100 nanometers. The fibers may include nanostructures, such as nanofibers, nanoparticles, nanospheres, nanopores, nanomicelles, and nano-roughness on surfaces. Nanostructures include structures ranging from approximately 1 nm to approximately 100 nm in at least one dimension. The nanostructures may be part of a nano-textured surface.

In an embodiment, the bone growth composition materials, such as the fibers or chips, may include nano-scale textured surfaces attractive to cells. Nano-scale textured surfaces provided on a bone matrix aid in growth factor retention, remodeling, cell attachment, and osteoconductivity of the bone matrix. In embodiments wherein the nanostructures of the textured surface comprise nanofibers, the nanofibers may be oriented. In some embodiments, the nanostructures of the textured surface may be biologically active. For example, the nanostructure may comprise biologically active biomolecules or incorporated with other biological factors such as peptides, growth factors, cytokines, DNA, RNA, siRNA etc.

In an embodiment, the nanoscale textured surface of bone is retained during processing steps to prepare the bone growth composition. In an embodiment, the nanoscale textured surface is imparted to bone or enhanced via one or more processing steps. In certain cases, the fibers are prepared without lyophilization or drying. Because removal of moisture can deteriorate surface nanostructures, maintaining wetted fibers as provided herein may be associated with improved nanoscale feature retention. However, in embodiments, even in cases where the fibers and/or chips remain wet through the process, the bone growth composition may be subjected to steps that improve nanoscale features on the bone surface, such as providing hydrogel carriers (e.g., dextran, pluronics, N,O-carboxymethylchitosan glucosamine) and/or adding nanoscale features as a surface coating or treatment via electrospinning nanofibers (e.g., collagen, polylactide, polycaprolactone, polyglycolide, chitosan, gelatin, or other nanofibrous material) onto bone surface. The nanofibers may include a pharmaceutical agent or bioactive material as provided herein.

As provided herein, surface area of the demineralized fibers was determined using the Brunauer, Emmett, and Teller (BET) technique. Results of the BET surface area measurements of the fibers prepared according to the disclosed techniques are shown in Table 1. Demineralized fibers were prepared using the techniques described herein. The fibers were dried using a supercritical carbon dioxide (sCO2) method. The sCO2 drying was performed as follows. Samples were pre-soaked in ethanol before being exposed to CO2 and ethanol for 2 hrs at 5000 psi/70° C. The vessel was then purged with CO2 and ethanol for 1 hr. Finally, the vessel was purged with CO2 for 2 hrs. Fibers prepared using the sCO2 method were compared to fibers prepared using alternative techniques such as freeze drying (e.g., lyophilization) or air drying in an oven at approximately 50° C. The BET surface area of the fibers was measured following the method in ISO 9277 with nitrogen (N2) gas. Any residual moisture in the samples was removed by placing the samples in a vacuum at 25° C. for 960 minutes, and the dry weight of the samples was recorded.

TABLE 1
BET surface area measurements of demineralized fibers.

| | | | BET | BET | BET |
|---|---|---|---|---|---|
| | Wet | Dry | Surface | Surface | Surface |
| | Mass | Mass | Area (cm²/g | Area (cm²/g | Area |
| Sample | (g) | (g) | dry) | wet) | (cm²/cm³) |
| sCO2 | 9.1797 | 0.4885 | 1,014,895 | 54,008 | 54,008 |
| Dried | | | | | |
| #1 | | | | | |
| sCO2 | 9.5925 | 0.5415 | 1,082,011 | 61,080 | 61,080 |
| Dried | | | | | |
| #2 | | | | | |
| Air-Dried | 10.103 | 0.452 | 2802 | 125 | 125 |
| Freeze-Dried | N/A | 0.454 | 665 | 30* | 30* |

*= For the freeze-dried sample, the wet mass was assumed to be 10 g to perform the BET surface area calculation.

As shown Table 1, the BET surface area of the demineralized fibers dried by sCO2 was 1,014,895 cm²/g and 1,082,011 cm²/g, respectively. Further, after normalizing the surface area of the demineralized fibers dried by sCO2 using the original wet mass, the surface area of the demineralized fibers dried by sCO2 is 54,008 cm²/g and 61,080 cm²/g, respectively. The density of wet fibers is approximately 1 g/cm³. This was determined by placing wet fibers with a known mass into a syringe with gradations to measure volume. Accordingly, the surface area of the fibers dried by sCO2 was 54,008 cm²/cm³ and 61,080 cm²/cm³, which is greater than the surface area of the fibers dried by freeze-dried and air dried-techniques.

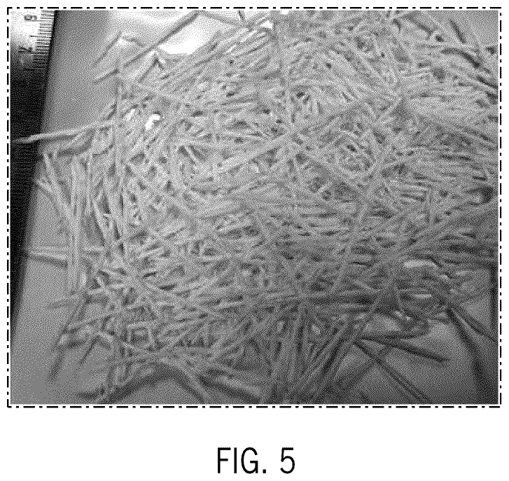

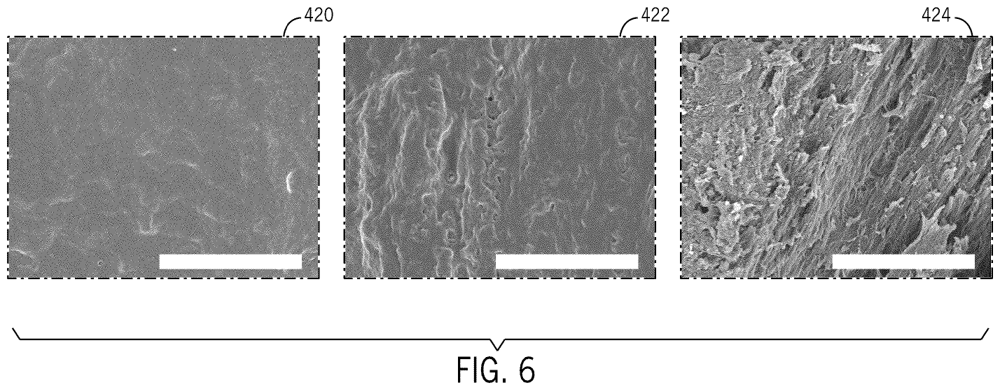

To further corroborate the BET surface area measurements, scanning electron microscopy (SEM) was performed. FIG. 6 shows SEM images of demineralized fiber samples that were freeze-dried, air-dried, or sCO2-dried. Accordingly, demineralized fibers that were freeze-dried, air-dried, or sCO2-dried were mounted on aluminum stubs and were sputter-coated with 60/40 Au—Pd at 2-3 nm thickness to increase conductivity. The samples were than evaluated using a Hitachi S3500N machine at an accelerating voltage of 5 k V. Representative images are shown at 2000× magnification (scale bar is 20 microns).

The freeze-dried fibers 420 have a relatively flat surface. The surface of the air-dried fibers 422 has some roughness and is not as smooth as the freeze-dried fibers 420. The sCO2-dried fibers 424 have extensive contouring and features that are noticeably different than the air-dried fibers 422 and freeze-dried fibers 420. As such, these SEM images confirm that the sCO2-dried fibers 424 have a higher surface area compared to the air-dried fibers 422 sample and freeze-dried fibers 422 sample. It is well established that sCO2-drying avoids the liquid-gas phase transition of air-drying and the liquid-solid/solid-gas phase transformations of freeze-drying that can alter the features of the material. Thus, sCO2-drying better preserves the native architecture of the demineralized fibers.

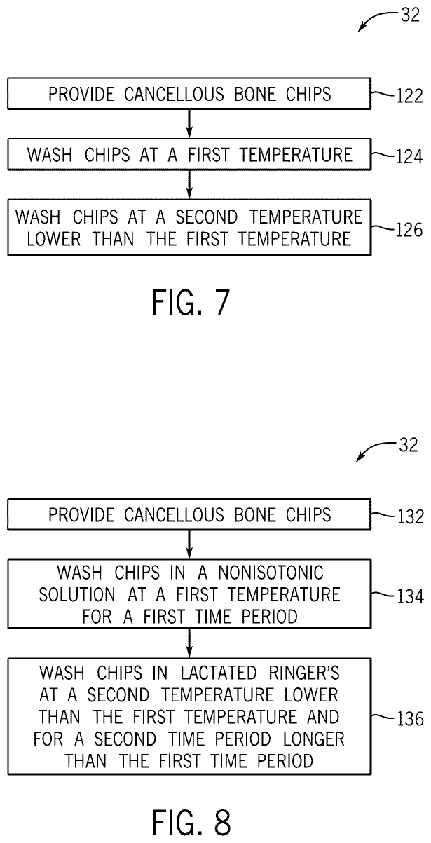

FIG. 7 is a two-step chip washing process 32 that may be used in conjunction with FIG. 1 or other bone handling processes. In an embodiment, the chip washing process 32 may be performed after milling a cancellous portion into chips. At step 122, cancellous bone chips are provided (e.g., from milling as in FIG. 1), and the chips are washed at a first temperature at step 124 and subsequently washed at step 126 at a second temperature lower than the first temperature. The first wash may be in a same or different solution than the second wash.

FIG. 8 is an embodiment of the two-step chip washing process 32 of FIG. 4. At step 132, cancellous bone chips are provided (e.g., from milling as in FIG. 1), and the chips are washed in a first washing solution that is a non-isotonic solution (e.g., water, filtered water) at a first temperature for a first time period at step 134 and subsequently at step 136 washed in a second washing solution that is Lactated Ringer's at a second temperature lower than the first temperature and for a second time period longer than the first time period (e.g., the first time period is shorter than the second time period). The illustrated process 32 is by way of example, and the time of washes may, in embodiments, be the same (e.g., 5-15 minutes), or the second wash may be shorter in time than the first wash in another embodiment.

In an embodiment, the first wash step is conducted at 40-50° C. (e.g., 42-44° C.) for 5-15 minutes in a closed vessel with agitation, spinning, and/or a motorized impeller. In an embodiment, the first wash step is conducted for a minimum of 5 minutes and a maximum of 15 minutes. The second wash may be conducted at 22-35° C. (e.g., 25° C.) for 5-20 minutes or at least 20 minutes in a closed vessel with agitation, spinning, and/or a motorized impeller.

The water used in the first wash is non-isotonic relative to the bone cells of the chips. The two-step wash maintains cell viability relative to a wash in an isotonic solution, which is an unexpected benefit.

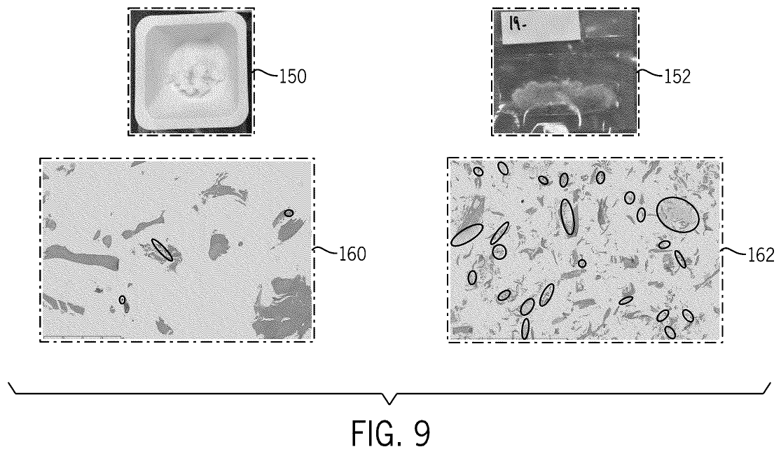

FIG. 9 shows a side-by-side comparison of a cancellous tissue portion sample 150 and associated histology image 160 washed with the two-step wash as discussed in FIG. 5 relative to a control cancellous tissue portion sample 152 and associated histology image 162 processed in a one-step wash. As shown in the image 160, the sample 150 has limited marrow and soft tissue attachment to chips while the image 162 is indicative of marrow and/or soft tissue attached to most chips in the control sample 152. The effectiveness of the two-step chip washing is demonstrated. Using this technique, there is very little marrow and soft tissue still attached to the bone chips after cleaning (left image 160). Very small infrequent pockets of non-bone tissue (circled in left image 160) can only be found at high magnification. When a single-step wash technique is used, considerable bone marrow and soft tissue still remain (circled in right image 162). Large pockets of non-bone tissue are evident throughout even at low magnification

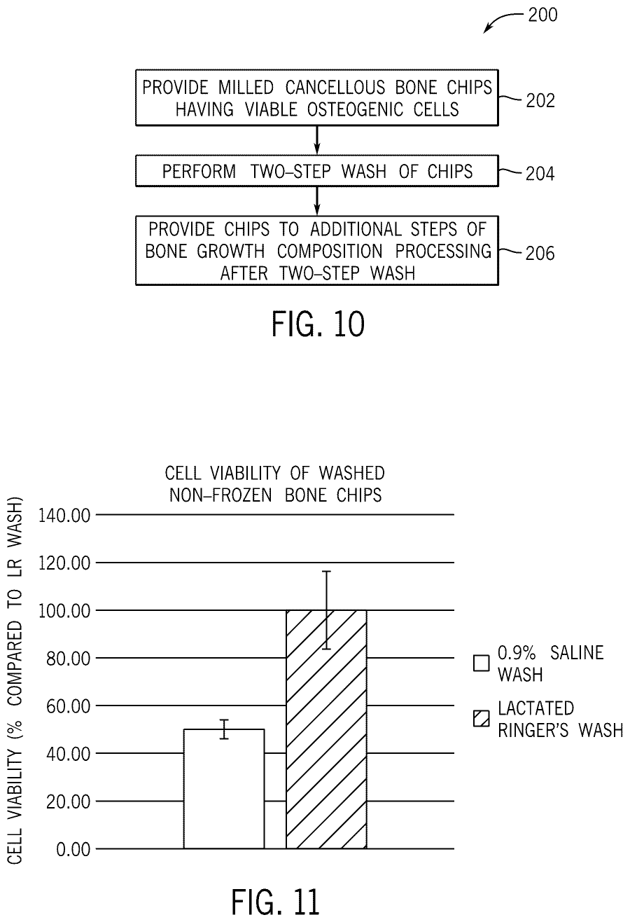

FIG. 10 is a method 200 of monitoring viability of osteogenic cells after a two-step washing process, as discussed with respect to FIGS. 4-5 and with reference to FIGS. 1-3. At step 202, milled cancellous bone chips are provided. The chips are washed using a two-step wash (see FIGS. 4-5) at step 204 as provided herein, and the washed chips are provided to additional steps of the bone growth composition processing at step 206.

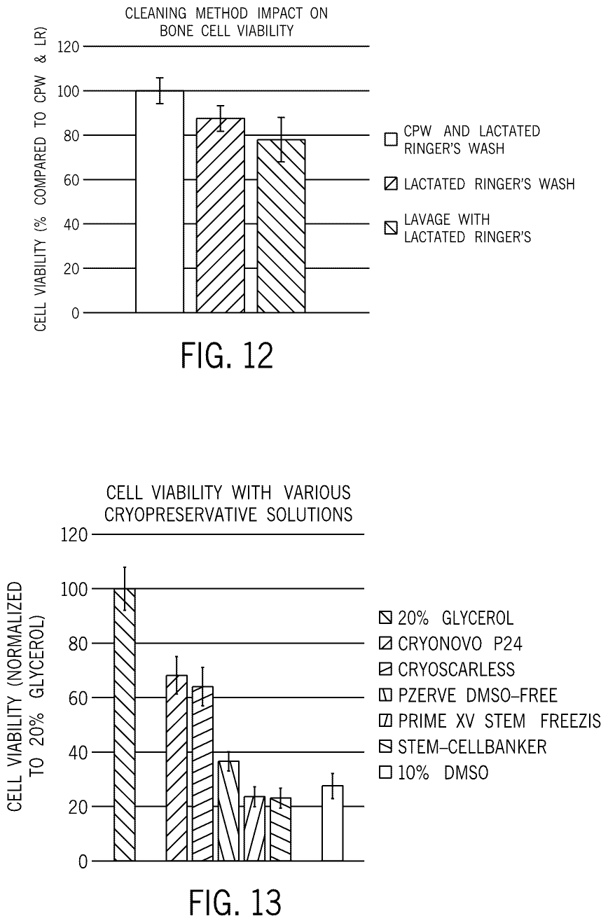

FIGS. 11-12 show example cell viability data for different washing conditions. FIG. 11 is a comparison of cell viability, as assessed by PrestoBlue, of washed non-frozen tissue using saline to wash non-frozen tissue using Lactated Ringer's from a single donor. Lactated Ringer's as a wash solution demonstrated improved viability relative to saline. The cell viability is shown as a percentage comparison to Lactated Ringer's of 0.9% saline for washed, non-frozen chips.

FIG. 12 is a comparison of cell viability, as assessed by PrestoBlue, of different washing procedures assessed for a single donor. A single-step Lactated Ringer's wash by stirring or a single-step MicroAire lavage with Lactated Ringer's, did not perform as well as a two-step water (CPW denoting a clean room water source or filtered water) and Lactated Ringer's wash. The cell viability is shown as a cell viability percentage compared to a two-step water and Lactated Ringer's wash for a single-step Lactated Ringer's wash or single-step MicroAire lavage with Lactated Ringer's in which the two-step wash showed improved viability.

The disclosed techniques may use a glycerol cryopreservative that can be used with direct freezing in certain embodiments. That is, packaged and sealed cellular bone matrix products may be placed directly in a conventional freezer, and do not necessarily require a controlled freezing process to maintain sufficient cell viability. In addition, in certain embodiments, the cellular bone matrix products as provided herein may be used without decanting liquid cryopreservative from the thawed product and/or without rinsing or centrifugation. In certain embodiments, the product is thawed to 37° C. in a solution bath, and the packaged and sealed cellular bone matrix products can remain in the cryopreservative at the thawing conditions for at least 2 hours or 4-6 hours after thawing without significant cell viability impacts.

In an embodiment, the frozen bone growth composition is in a solid state and transitions to a partially liquid state after thawing. That is, certain portions of the bone growth composition may remain solid while certain portions may be liquid. In an embodiment, the bone growth composition, after thawing, may have a flowable, slurry, or putty-like consistency. The term “flowable” may refer to compositions whose consistencies range from those which can be described as shape-sustaining but readily deformable, e.g., those which behave like putty, to those which are runny. Specific forms of flowable bone powder compositions include cakes, pastes, creams and fillers. Reference is made to U.S. Pat. No. 5,290,558, herein incorporated by reference in its entirety, for discussion of flowable materials. The bone growth composition may be a gel, putty, paste, cake, or solid in embodiments. The disclosed bone growth composition provides a product with sufficient cohesion for efficient manipulation of the product intraoperatively and, in embodiments, improved mixing with other materials (autograft, other bone graft materials, etc.), and improved bone defect filling and shaping to optimize bone defect repair.

FIG. 13 shows a comparison of cell viability for a glycerol cryopreservative in Lactated Ringer's (e.g., 80% Lactated Ringer's) relative to certain commercial cryopreservative solutions demonstrating that 20% glycerol by volume outperforms multiple commercial cryopreservatives under the same conditions. The cell viability is shown as a cell viability comparison normalized to the 20% glycerol by volume results for various other cryopreservative solutions.

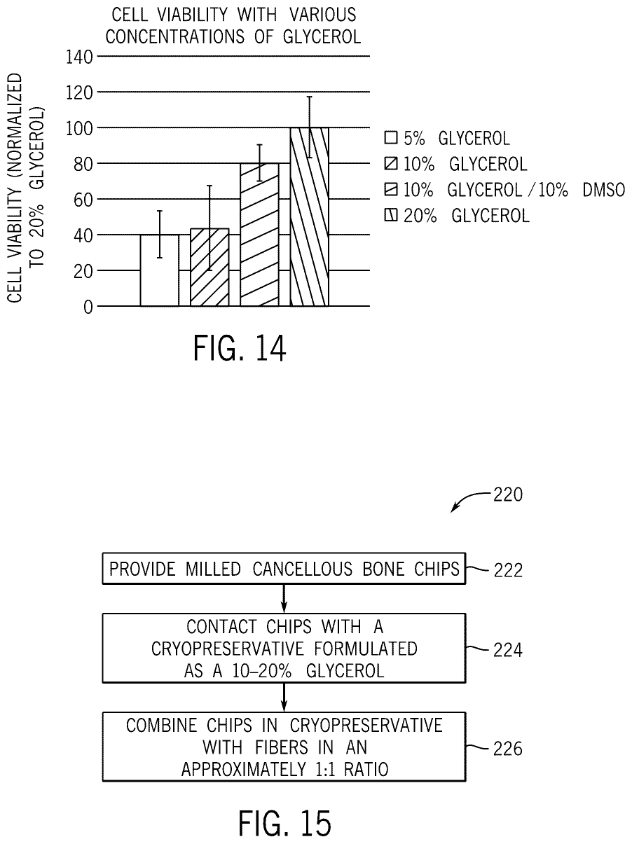

FIG. 14 shows cell viability for various glycerol concentrations in Lactated Ringer's. The cell viability is shown as a cell viability comparison for various glycerol concentrations in Lactated Ringer's normalized to 20% glycerol by volume.

FIG. 15 is a method 220 of combining chips and fibers during cellular bone matrix production and with reference to FIGS. 1-5. At step 222, milled cancellous bone chips are provided. At step 224, chips are contacted with a cryosolution or cryopreservative as provided herein. In certain embodiments, the cryopreservative contact occurs subsequent to the antimicrobial contact and rinse. In certain embodiments, the rinse is Lactated Ringer's, and the rinse solution is removed to facilitate the cryopreservative soak in the rinse solution with 10-20% glycerol by volume. In other embodiments, the glycerol may be directly added to the rinse solution. The soak may be conducted at 22-30° C. in an embodiment.

After the appropriate amount of time has elapsed (e.g., a minimum of 60 minutes to a maximum of 180 minutes in an embodiment), the excess cryopreservative solution of the chips can be decanted, leaving enough solution behind to keep the combined cellular bone matrix product wetted. At step 226, the chips and fibers are combined in an approximate 1:1 ratio. By way of example, chips and fibers can be aliquoted using a same-sized volume (e.g., cubic centimeters) measure. The combined material can be filled into various pouches according to desired size and sealed. The chips and fibers, together with the cryopreservative solution, may be measured according to total volume, such as cubic centimeters. In an embodiment, the liquid of the cryopreservative solution may be measured in mL. For example, a bone growth composition may be aliquoted using 1-15 cubic centimeters of fibers and 1-15 cubic centimeters of chips. The chips and fibers may carry liquid that, in the packaging, forms the cryopreservative solution. This solution may be present in liquid volume amounts of 1-10 mL (e.g., 1-50 cc), in an embodiment. In an embodiment, the excess (e.g., free or decantable) total liquid amount in the packaging is 0-2 mL and a total amount of the fibers or chips is 1-10 cubic centimeters or 6-8 cubic centimeters.

It should be understood that the disclosed compositions are wetted and, therefore, the cryopreservative solution that is present may be associated with, absorbed by, or otherwise coupled together with the bone tissue. Thus, more liquid may be present than is decantable within the package. In contrast to less viable compositions, the disclosed wetted bone growth compositions are exposed to associated cryopreservative solution, even after thawing, and nonetheless maintain significant viability.

In an embodiment, any remaining solution in the combined chips and fibers composition has a cryopreservative, e.g., glycerol, present in a range of 1-25%. In certain embodiments, the fibers, stored in Lactated Ringer's, are drained prior to combination. Thus, little or no Lactated Ringer's from the stored fibers may be carried over into the combined composition, and a majority of the solution present is the cryopreservative solution. Accordingly, chips in a cryopreservative solution having 20% glycerol may yield a cellular bone matrix composition having 1-20% glycerol in an embodiment. In certain embodiments, the Lactated Ringer's solution is present in about equal volume as the cryopreservative solution. By way of example, in a 1:1 ratio combination, the existing cryopreservative is halved in percentage after combination. Accordingly, a cryopreservative solution having 20% glycerol has about 10% glycerol after combination in such an embodiment. In certain embodiments, the fibers are retained in Lactated Ringer's prior to combination while the chips have been drained or partially drained. Accordingly, a cryopreservative solution having 20% glycerol has less than 10% glycerol after combination in such an embodiment.

The disclosed cellular bone matrix production techniques incorporate a cryopreservative contact step to maintain cell viability at cryogenic temperatures. However, cryopreservative agents, while protective at cryogenic temperatures, may be cytotoxic at temperatures higher than the cryogenic temperatures. Thus, cryopreservatives have potential to reduce cell viability both during the freezing process as well as during the thawing process. In addition, the time spent in contact with the cryopreservative after thawing is dependent on the end user preparation conditions and preferences. Certain techniques may involve a decanting and/or centrifugation step post-thawing to remove the cryopreservative present in the thawed sample. However, these steps may also be associated with loss of viable cells to handling. In addition, certain techniques may also involve a controlled freezing process that involves a specialized freezing container with a linear temperature decrease per minute to reduce cell loss as a result of cryopreservative contact during the freezing process.

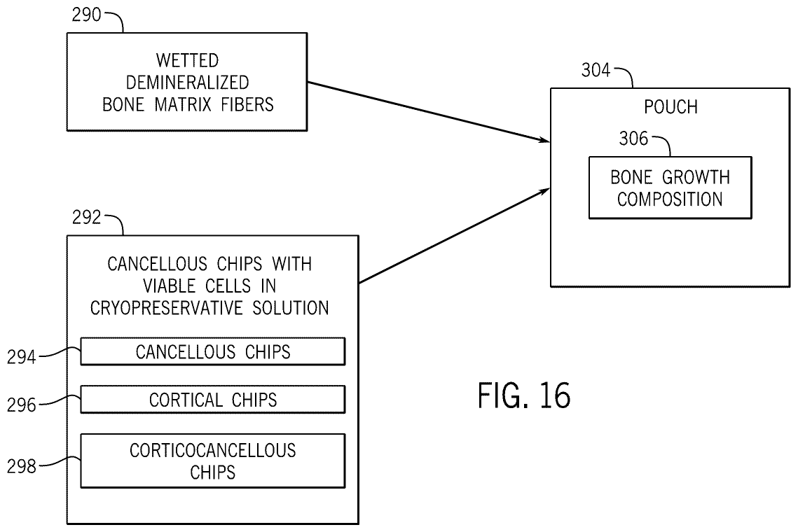

FIG. 16 shows an example bone growth composition product in which wetted demineralized bone matrix fibers 290 are combined with a cancellous chip mixture in cryopreservative solution 292 having viable cells that are in cryopreservative solution. As discussed herein, the cancellous chips in cryopreservative solution 292 may include a mix of cancellous chips 294, cortical chips 296, and corticocancellous chips 298. The chips 292 may be predominantly cancellous chips 294 (e.g., 50-90% or greater) with the remainder being cortical chips 296 and/or corticocancellous chips 298.

The fibers 290 and cancellous chips in cryopreservative solution 292 are packaged in a container, shown as a pouch 304, to form a cryo-ready bone growth composition 306. The pouch 304 is sealed, and the bone growth composition 306 is frozen, stored, and/or transported for use. Thus, the bone growth composition product may, in embodiments, include both the packaging, such as the pouch 304, and the bone growth composition 306.

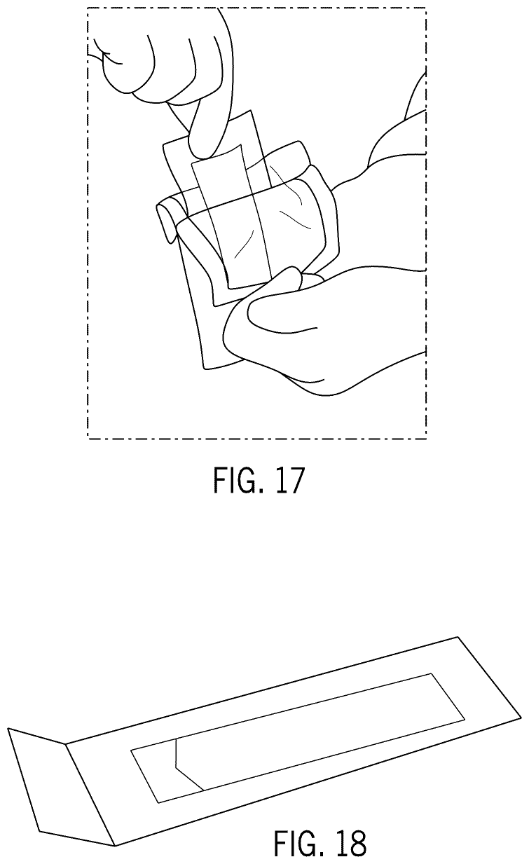

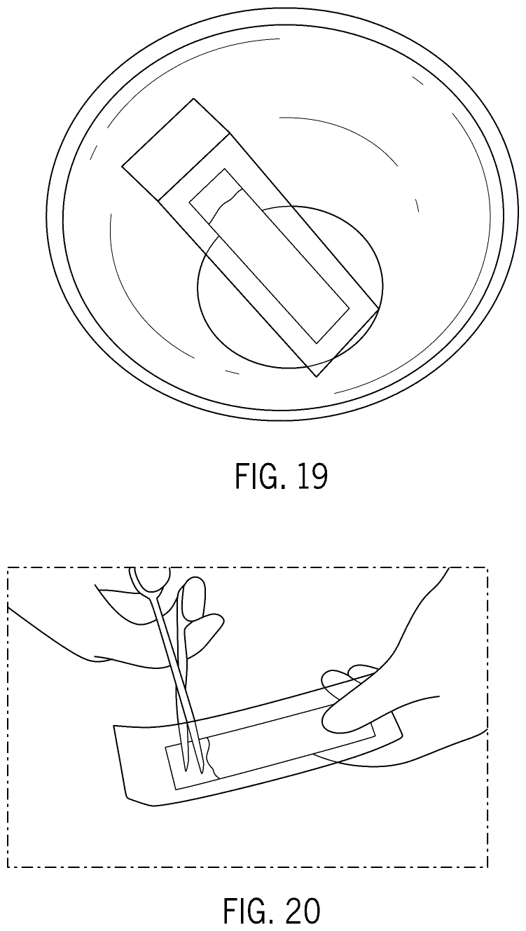

FIG. 17 is an image of an example packaging including a scaled interior pouch with bone growth composition being aseptically removed from a protective outer packaging. FIG. 18 shows the sealed interior pouch with bone growth composition. FIG. 19 shows thawing of bone growth composition in the sealed interior pouch. FIG. 20 shows an end user opening the interior pouch after thawing to access the bone growth composition.

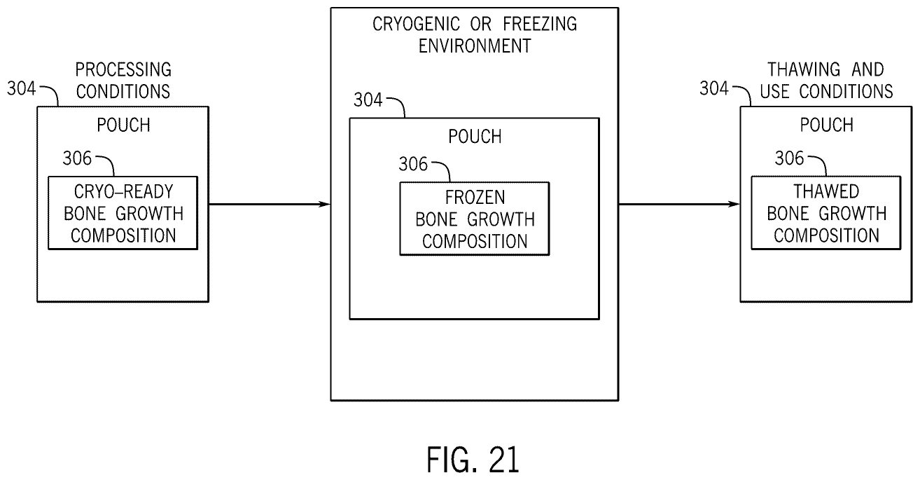

FIG. 21 shows the bone growth composition packaging in different conditions. At the end of processing of the donor tissue, as shown in FIG. 16, the fibers 290 and chips 292 (e.g., chip mixture) are combined to form the cryo-ready bone growth composition 306 that is packaged and sealed in the pouch 304. The processing conditions may be room temperature conditions, e.g., 20-25° C. After packaging, the pouch is placed in a freezing environment, e.g., a cryogenic environment, and the bone-growth composition 306 in the pouch transitions from a room temperature or nonfrozen state to a frozen bone growth composition 306. When the bone growth composition 306 is ready for use in a patient, the pouch 304 is removed from the freezing environment and thawed, and the frozen bone growth composition 306 transitions to a thawed bone growth composition 306.

In an embodiment, the number of viable cells of the bone growth composition 306 present at the time of packaging is reduced by less than 30% or less than 50% after thawing.

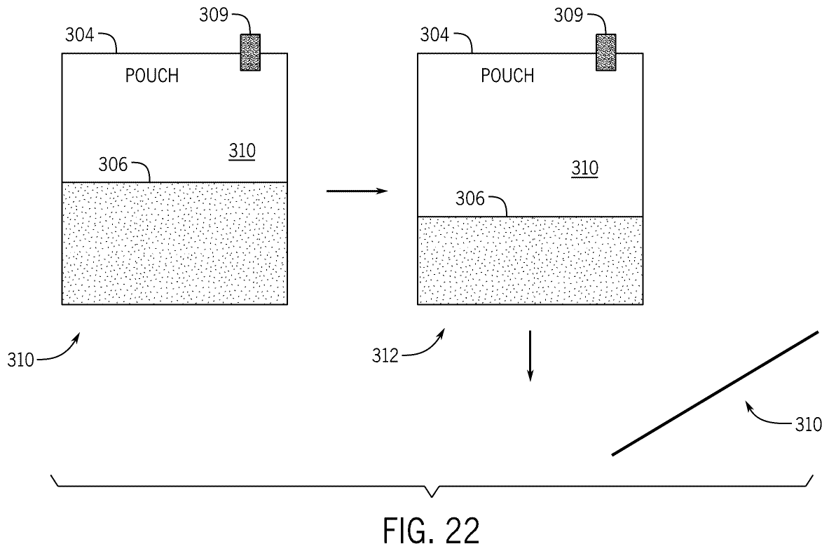

FIG. 22 shows a bone growth composition packaging system in which the pouch 304 includes features to permit decanting of liquid via a port 309 that, when opened, breaks the pouch seal. Additionally or alternatively, the pouch 304 may include a release liner 310 that can be peeled away or removed to reveal a perforated wall 312 including perforations that extend from an exterior of the pouch 304 through to an interior space 310 to permit draining of cryopreservative liquid via the perforations. The perforations may be sized to retain chips and fibers in the pouch 304 while permitting liquid drainage.

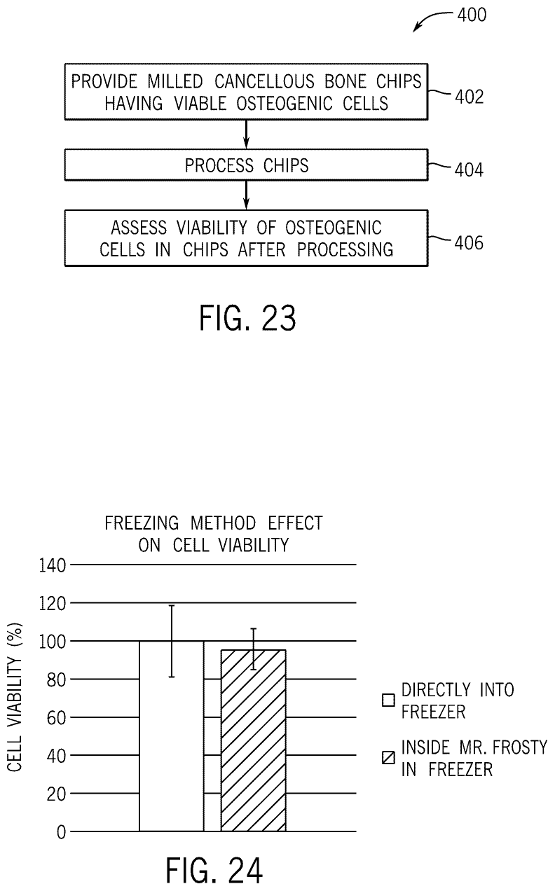

As provided herein, osteogenic may refer to the ability of a material to enhance or accelerate the growth of new bone tissue by one or more mechanisms such as osteogenesis, osteoconduction, and/or osteoinduction. FIG. 23 is a method 400 of monitoring viability of osteogenic cells after chip processing. At step 402, milled cancellous bone chips are provided. The chips are processed at step 404 as provided herein, and the viability of the osteogenic cells is assessed at step 406. In the illustrated embodiment, the viability is assessed after processing, e.g., after the chips are contacted with the cryopreservative. Viability may additionally be assessed following contact with the antimicrobial agent and/or after thawing.

In an embodiment, the viability is assessed using a modified PrestoBlue assay. Viable cells are able to convert resazurin (blue) to resorufin (pink). The absorbance indicative of pink color conversion can be compared against a standard curve based on known numbers of viable cells to estimate a viable cell count in a sample.

As provided herein, the cancellous chips may have an estimated viable cell concentration of at least 50,000 viable cells/cc.

Osteoinductivity, may refer to the quality of being able to recruit cells from the host that have the potential to stimulate new bone formation. Any material that can induce the formation of ectopic bone in the soft tissue of an animal is considered osteoinductive. For example, most osteoinductive materials induce bone formation in athymic rats when assayed according to the method of Edwards et al., “Osteoinduction of Human Demineralized Bone: Characterization in a Rat Model,” Clinical Orthopaedics & Rel. Res., 357:219-228, December 1998. In other instances, osteoinduction is considered to occur through cellular recruitment and induction of the recruited cells to an osteogenic phenotype. Osteoinductivity score refers to a score ranging from 0 to 4 as determined according to the method of Edwards et al. (1998) or an equivalent calibrated test. In the method of Edwards et al., a score of “0” represents no new bone formation; “1” represents 1%-25% of implant involved in new bone formation; “2” represents 26-50% of implant involved in new bone formation; “3” represents 51%-75% of implant involved in new bone formation; and “4” represents >75% of implant involved in new bone formation. In most instances, the score is assessed 28 days after implantation. However, the osteoinductivity score may be obtained at earlier time points such as 7-, 14-, or 21-days following implantation. Percentage of osteoinductivity refers to an osteoinductivity score at a given time point expressed as a percentage of activity, of a specified reference score.

As provided herein, the cellular bone matrix thawed from frozen may have an osteoinductivity score of at least 1 or at least 2 based on a 50/50 chip vs. fiber formulation. Results of osteoinductivity tests for cellular bone matrix products prepared according to the disclosed techniques are shown below in Table 2. The osteoinductivity tests were performed as in Edwards et al. and using a rat muscle pouch assay. Table 3 demonstrates that the osteoinductivity of the tissue was maintained during extended storage at −70° C.

TABLE 2
Osteoinductivity scores

| Description | OI Score |
|---|---|
| 50% chips/50% fibers | 1.49 ± 0.66 |
| (n = 9 lots) | |
| 100% fibers | 2.96 ± 0.62 |
| (n = 4 lots) | |

TABLE 3
Osteoinductivity is maintained following storage at −70 C.

| Group | Description | Original Score | Re-test Score |
|---|---|---|---|
| 1 | 50% chips/ | 2.25 | 2.00 (after 1 year) |
| | 50% fibers | | |
| 2 | 50% chips/ | 2.13 | 2.88 (after 1 year) |
| | 50% fibers | | |
| 3 | 50% chips/ | 2.00 | 2.63 (after 1 year) |
| | 50% fibers | | |
| 4 | 50% chips/ | 0.75 | 1.25 (after 1 year) |
| | 50% fibers | | |
| 5 | 100% fibers | 3.50 | 3.25 (after 1 year) |
| 6 | 100% fibers | 3.00 | 3.50 (after 6 months) |

In the testing conditions, samples were stored in the freezer at −70 C for an extended amount of time (shown in table 3) and tested again in the rats. The results confirm that the tissue retained its osteoinductivity. On average, the osteoinductive score for the disclosed cellular bone matrix compositions is higher than that of tested competitive products. By way of example, OI scores were assessed for competitive products as follow: OI for Osteocel Pro (n=3 lots) was 0.9±0.1. For OsteoCel Plus (n=5 lots), it was 0.2±0.3. Map3 (no longer on market) was 0.25±0.0 (n=3 lots).

The disclosed cellular bone matrix compositions are associated with relatively higher viable cell counts and longer shelf lives and/or longer time windows for use after thawing as a result of improved processing. In one example, disclosed cellular bone matrix products maintain viability for at least 2 hours or at least 4-6 hours after thawing and without separation from the cryopreservative solution. In an embodiment, the total thaw time for a 15 cubic centimeter (as packaged) size is 6 minutes or less.

FIG. 24 is a percentage comparison between the direct and controlled freezing showing very similar viability results. As shown, the use of a controlled freezing device provided no cell viability benefit under the assessed conditions. Accordingly, in certain embodiments, the pouches or other containers may be placed directly in cryogenic conditions, such as a −70° C. freezer, without use of the controlled freezing device. A benefit of the disclosed production techniques for cellular bone matrix compositions is a less complex and more user-friendly freezing process.

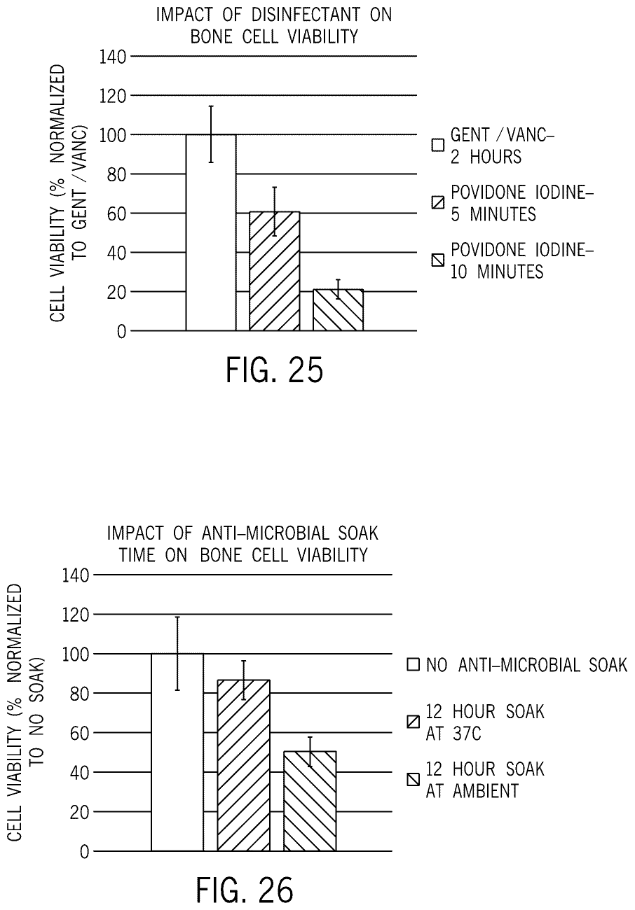

FIGS. 25-28 show results from different antimicrobial agent soaks and contact conditions. FIG. 25 shows results of different soak times and antimicrobial agents, such as a gentamicin and vancomycin or 1% povidone-iodine. The cell viability is shown as with results normalized to a 2-hour gentamicin and vancomycin soak. The presence of povidone iodine was associated with decreased cell viability.

FIG. 26 shows benefits of performing the antimicrobial contact step at temperatures above room temperature. A 12-hour antimicrobial soak at 37° C. demonstrated elevated results relative to room temperature soaks at various lengths. The cell viability is shown normalized to a no antimicrobial contact control.

The effectiveness of the antibiotic/antimycotic soak in eradicating bioburden was demonstrated by spiking cancellous bone chips with a representative panel of microorganisms and measuring the kill efficiency. As shown in the Table 4, the antimicrobial cocktail was highly effective against a broad range of bacteria and yeast with 3-7 log reductions observed.

| | | Average Log | Average % |
|---|---|---|---|
| | Microbe Type | Reduction | Reduction |
| Staphylococcus aureus | Gram+ cocci | >7 | 99.999973 |
| (ATCC #6538) | | | |
| Enterococcus faecalis | Gram+ cocci | 5 | 99.99957 |
| (ATCC #4082) | | | |
| Pseudomonas | Gram− rod | 6 | 99.999939 |
| aeruginosa | | | |
| (ATCC #9027) | | | |
| Corynebacterium | Gram+ rod | >7 | 99.999971 |
| xerosis | | | |
| (ATCC #373) | | | |
| Bacillus atrophaeus | Gram+ rod (spore- | 3 | 99.86 |
| (ATCC #9372) | former) | | |
| Clostridium | Anaerobe | 4 | 99.9943 |
| sporogenes | (spore- | | |
| (ATCC #11437) | former) | | |
| Candida albicans | Yeast | 7 | 99.999988 |
| (ATCC #10231) | | | |
| Aspergillus brasiliensis | Mold | 0 | N/A |
| (ATCC #16404) | | | |

Table 4 displays the effectiveness of the antibiotic soak in eradicating bioburden.

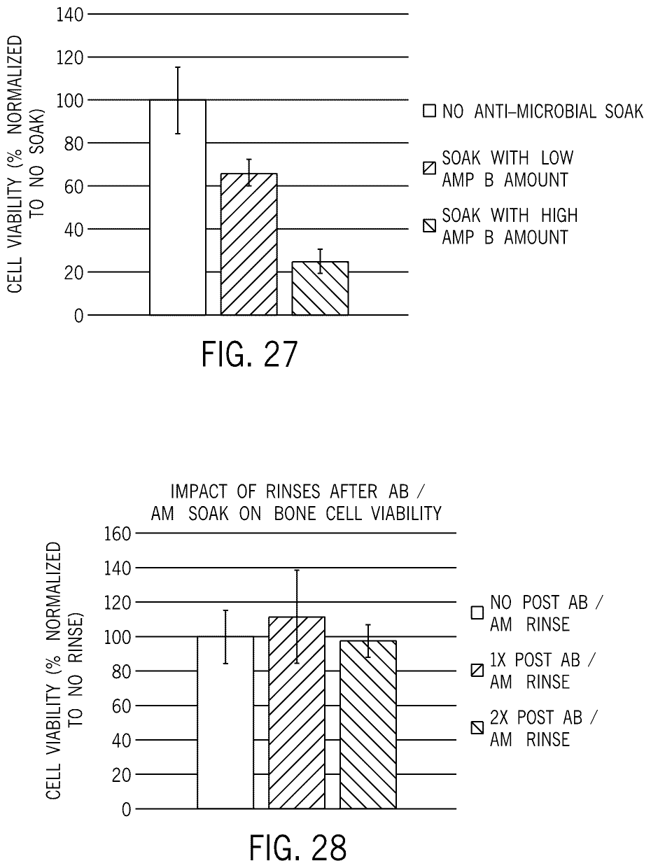

FIG. 27 shows results of different soak concentrations. A concentration-dependent decrease in bone cell viability was observed.

FIG. 28 shows effects on cell viability using different post-antimicrobial soak rinse conditions results relative to a no rinse control. Including at least one rinse step did not reduce cell viability.

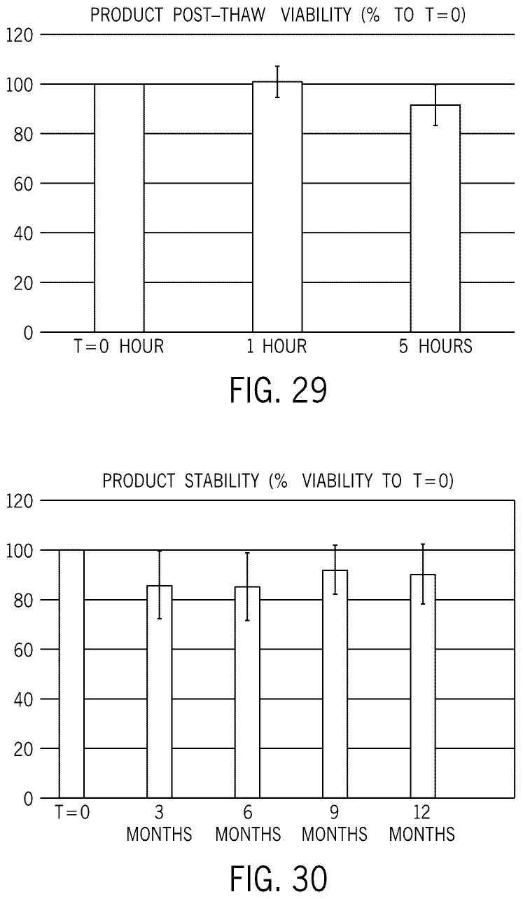

FIG. 29 shows post-thaw viability data for the bone growth composition of approximately, 100% at 1 hour and 90% at 5 hours normalized to the number of cells viable immediately after thawing (time zero or T=0). FIG. 30 shows post-thaw viability data for the bone growth composition of approximately 90% after 3 months, 6 months, 9 months, and 12 months of storage at −70° C. normalized to the number of cells when the tissue was first placed into the freezer at the end of processing (time zero or T=0).

With the foregoing in mind, bone growth composition (i.e., cellular bone matrix tissue) generated and treated using the techniques as discussed herein was stored in packaging at roughly −75° C. The cellular bone matrix, when removed, was thawed by placing the inner pouch with tissue in a water bath at 37° C. After approximately five minutes, no cold or hard spots could be detected indicating that thawing was complete.

The thawed cellular bone matrix tissue was then placed in 4 mg/mL of Type II Collagenase digestion solution (in Alpha MEM media). The sample was placed in an incubator shaker set at 200 rpm for 45 minutes at 37° C. to release the cells. After 45 minutes, the solution was poured through 70 μM cell strainers to remove large pieces of bone debris. Fresh collagenase solution was added to the cellular bone matrix samples for another 45 minutes, after which the digestion fluid was strained. After each digestion, the suspension was centrifuged to form a cell pellet.

Labeling for flow cytometry was performed. DAPI stain was used to identify nucleated cells. CD45 was included as a pan-leukocyte marker expressed by white blood cells. Labeled samples were analyzed. This evaluation (digestion and flow cytometry) was performed twice. During the second round of testing, freeze-dried bone which had been thoroughly cleaned (Medtronic SpinalGraft part number 100015, irradiated human cancellous chips, 1-4 mm) and human blood (Innovative Research, Ref #IWB1K2E10 ML) were included as controls.

| | | Total Nucleated Cell |
|---|---|---|
| Sample | CD45+ Events | Events |
| CBM #1 | 657 | 9,833 |
| CBM #2 | 490 | 8,128 |
| Freeze-Dried Bone Chips | 53 | 96 |
| Blood | 19,074 | 19,784 |

Table 5 displays the results of the flow cytometry analysis.

Accordingly, Table 5 displays the results of the flow cytometry analysis. CD45+ cells were detected in the cellular bone matrix samples at a low frequency compared to the total nucleated cells. In the first cellular bone matrix sample (e.g., CBM #1), the CD45+ events were 6.65% of the total nucleated cell events. This percentage was 6.03% for the second cellular bone matrix sample (e.g., CBM #2). A small number of events was measured for the freeze-dried bone chips indicating minimal background noise in the method. As expected, almost all of the nucleated cells in the blood sample (e.g., blood) were CD45+ leukocytes. Leukocytes are key members of the immune system with the ability to activate both innate and humoral immune responses and are immunogenic. These data indicate that a low level of immunogenic CD45+ cells may be present in the cellular bone matrix prepared using the techniques discussed herein.

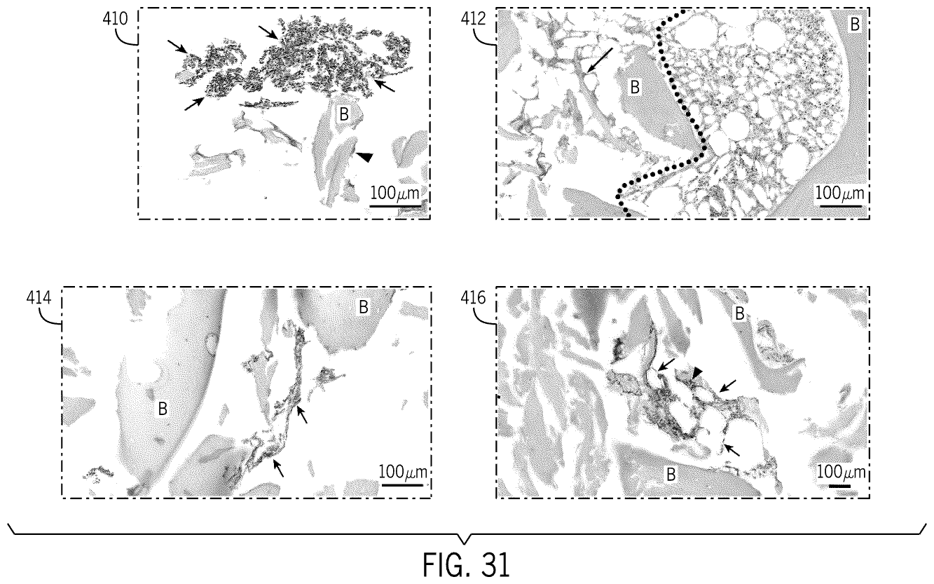

Further, final cellular bone matrix products from three separate donors (e.g., samples from Donor 1, Donor 2, Donor 3) were subjected to histological processing. The samples were demineralized in EDTA, embedded in paraffin, and stained with H&E. The slides were reviewed by a trained histopathologist. As shown in FIG. 31, small amounts of residual marrow elements and hematopoietic cells were detected in the samples from the three donors, as demonstrated in histology images 410, 412, 414, and 416. This includes red blood cells (erythroid) and white blood cells (myeloid and lymphoid). Image 410 (e.g., sample from Donor 1) shows clusters of bare nuclei (potential lymphoid precursors) were occasionally noted scattered throughout samples (as indicated by arrows). “B” refers to bone. Image 412 (e.g., sample from Donor 1) shows a dotted line, wherein to the right of the dotted line the bone marrow contained clearly identifiable hematopoietic cells (both erythroid and myeloid precursors). Further, the area to the left of the dotted line shows that bone marrow structures were still present; however, only few, scattered, potential hematopoietic cells are observed (arrow). Image 414 (e.g., sample from Donor 2) shows non-mineralized tissue (potentially residual marrow elements) are present (indicated by arrows) between pieces of bone (as indicated by “B”). Image 416 (e.g., sample from Donor 3) shows non-mineralized tissue with mostly basophilic material (arrows) was detected between pieces of bone (as indicated by “B”), and occasionally, potentially viable/intact cells were observed (as indicated by arrowhead). As such, these histology results further demonstrate that cellular bone matrix prepared as discussed herein contains a low amount of potentially immunogenic donor-derived blood cells.

With the foregoing in mind, Table 6 shows the amount of residual calcium in the demineralized fibers after drying measured using atomic absorption spectroscopy. As seen in Table 6, very little calcium remains following the demineralization procedure. The residual calcium values are well below 8%, indicating the fibers are fully demineralized per AATB standards.

| | Sample 1 | Sample 2 | Sample 3 | Average |
|---|---|---|---|---|
| | (% Calcium) | (% Calcium) | (% Calcium) | (% Calcium) |
| Donor 1 | 0.755 | 0.357 | 0.862 | 0.658 |
| Donor 2 | 0.019 | 0.018 | NA | 0.019 |
| Donor 3 | 0.042 | 0.656 | NA | 0.349 |
| Donor 4 | NA | NA | NA | 0.022 |
| Donor 5 | NA | NA | NA | 0.047 |
| Donor 6 | NA | NA | NA | 0.186 |
| Donor 7 | NA | NA | NA | 0.021 |
| Donor 8 | NA | NA | NA | 0.002 |

Table 6 displays the percent of calcium measured in demineralized fibers.

With the preceding in mind, the disclosed techniques provide improvements in maintaining the cells, tissues, or other biologically active components of a bone growth composition from harvesting through processing and until the composition is readied for transplantation into a recipient. During the interval between harvest and implantation, it is also desirable to monitor and control the environmental conditions, and storage parameters to maintain the integrity, viability, and biochemical activity of the harvested and processed biological material from which the bone growth composition is formed.

Thus, the disclosed bone growth compositions have improved viability and/or biologic activity. While it is contemplated that the parameters of storage temperature, atmospheric pressure, ambient environmental conditions, and such like will provided to users of the bone growth composition, the bone growth compositions have a wider tolerance of storage and handling conditions to reduce the effects of end user variability and preferences on the therapeutic effectiveness of the disclosed bone growth compositions. For example, not all end users have access to stringent storage conditions, such as a −70° C. freezer. However, the disclosed bone growth compositions maintain suitable activity in a conventional −20° C. freezer. The bone growth composition has been demonstrated to be viable (e.g., greater than 80% viability) measured after 5 days at −20° C. In particular, viability was observed to be 100% after 5 days at −20° C. and 96% after 15 days at −20° C. In addition, viability was observed to be 95% after 4 weeks at −40° C. and 71% after 8 weeks at −40° C. Further, because freezers may be subject to variability in operating conditions, the disclosed bone growth compositions are able to maintain sufficient viability through fluctuations in freezer operating conditions, such that intermittent changes of temperature within a tolerance band (e.g., −15° C. to −25° C.) will not significantly alter the therapeutic effectiveness of the disclosed bone growth compositions.

The disclosed techniques are directed to bone growth compositions and methods. In certain embodiments, the bone growth compositions and methods may be cryogenically preserved. In an embodiment, cryogenic preservation, e.g., freezing, refers to exposing a population of cells, tissues, or organs to a cryogenic environment. A cryogenic environment may refer to temperatures below 0° C., below −20° C., below −70° C. In an embodiment, a cryogenic environment may be between about 0° C. to about −200° C.

A cryoprotectant or cryopreservative may refer to agents or materials that prevents or reduces undesirable damage to a biological material caused by lowering the temperature of a biological material or any substance or material which enhances, strengthens or otherwise increases the ability of the biological material to withstand lowered temperatures. As disclosed herein, the bone growth compositions and associated cryopreservation methods may include the addition of one or more cryoprotectants or cryopreservative compounds to permit freezing of the sample, and/or maintenance of the sample at temperatures generally below 0° C. Exemplary cryoprotectants and/or cryopreservative compounds, as used in the context of the disclosed techniques may include, but are not limited to, ice-suppressing cryoprotectants (e.g., non-colligative agents such as Supercool X-1000™ and Supercool Z-1000™, 21^{st} Century Medicine, Rancho Cucamonga, Calif.) glycerol, dimethylsulfoxide (DMSO), ethylene glycol, propylene glycol, polyethylene oxide (PEO), acetamide, ethanol, methanol, butanediol, carbohydrates (including sugars such as glucose, fructose, dextrans, sucrose, lactose, and trehalose), polyvinyl alcohols, hydroxyethyl starch, serum albumin. With CBMs, cryopreservation ensures the osteogenic potential of allogeneic cells providing benefits for the bone grafting site.

In certain embodiments, steps that involve freezing and/or thawing of a tissue sample or cell population may be achieved by, e.g., bringing the temperature of a refrigerated tissue or cell sample down to a suitable sub-zero temperature, or alternatively, bringing the temperature of a sub-zero stored sample up to refrigerated (and, optionally, to either room or recipient body temperature immediately prior to implantation). Freezing may include direct freezing a room temperature composition. Such steps in the disclosed methods may employ submersion vessels or frozen storage means to prepare the frozen tissue or cell sample, while conventional means such as a heated water bath or such like device, submerging the packaged frozen sample into a sample of growth medium, biological buffer, or tissue/organ storage solution (e.g., pre-warmed to the desired temperature), may be employed to bring the temperature of a frozen tissue sample to the desired temperature required for transplanting the bone growth compositions into the body of a suitable recipient.

It should be understood that exposure to a cryogenic environment may result in the freezing of liquid portions of a bone growth composition. However, the resultant bone growth composition may include composite structures that do not change in state between freezing and thawing and/or that change state at different temperatures relative to water in the sample. Thus, in an embodiment, a bone growth composition as provided herein may be referred to as frozen or may freeze at temperatures that cause liquid portions of the composition to transition to a solid state.

As provided herein, antimicrobial includes, for example, antibiotics, antifungal, antiviral agents or the like. Antimicrobial agents to treat infection include by way of example and not limitation, antiseptic agents, antibacterial agents; polyene antifungals (e.g., amphotericin B), quinolones and in particular fluoroquinolones (e.g., norfloxacin, ciprofloxacin, lomefloxacin, ofloxacin, etc.), aminoglycosides (e.g., gentamicin, tobramycin, etc.), glycopeptides (e.g., vancomycin, etc.), lincosamides (e.g., clindamycin), cephalosporins (e.g., first, second, third generation) and related beta-lactams, macrolides (e.g., azithromycin, erythromycin, etc.), nitroimidazoles (e.g., metronidazole), penicillins, polymyxins, tetracyclines, or combinations thereof. Some exemplary antimicrobial agents include, by way of illustration and not limitation, acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chloroxylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; chlorhexidine, cloxyquin; colistimethate sodium; colistin sulfate; coumermycin; coumermycin sodium; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; cpitetracycline hydrochloride; crythromycin; crythromycin acistrate; crythromycin estolate; crythromycin cthylsuccinate; erythromycin gluceptate; crythromycin lactobionate; crythromycin propionate; erythromycin stearate; ethambutol hydrochloride; ethionamide; fleroxacin; floxacillin; fludalanine; flumequine; tromethamine; fumoxicillin; furazolium chloride; furazolium tartrate; fusidate sodium; fusidic acid; ganciclovir and ganciclovir sodium; gentamicin sulfate; gloximonam; gramicidin; haloprogin; hetacillin; hetacillin potassium; hexcdine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine sodium; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodium; nafcillin sodium; nalidixate sodium; nalidixic acid; natainycin; nebramycin; ncomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillins such as penicillin g benzathine, penicillin g potassium, penicillin g procaine, penicillin g sodium, penicillin v, penicillin v benzathine, penicillin v hydrabamine, and penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; zorbamycin; or combinations thereof.

Certain embodiments of the disclosure are discussed in the context of cellular bone matrices or bone matrices. However, it should be understood that one or more compositions or processing steps may be used for other bone growth compositions. For example, the disclosed process steps may be used alone or in combination with other type of bone growth processing that includes one or both of chips or demineralized fibers.

The disclosed bone growth compositions may include a bioactive or pharmaceutical agent, which may be disposed in, packaged with, coated on or combined with the bone growth composition. The term “bioactive agent” as used herein is generally meant to refer to any substance that alters the physiology of a patient. The term “bioactive agent” may be used interchangeably herein with the terms “therapeutic agent,” “therapeutically effective amount,” and “active pharmaceutical ingredient”, “API” or “drug”.

Bioactive agent or bioactive compound is used herein to refer to a compound or entity that alters, inhibits, activates, or otherwise affects biological or chemical events. For example, bioactive agents may include, but are not limited to, osteogenic or chondrogenic proteins or peptides, anti-AIDS substances, anti-cancer substances, antibiotics, immunosuppressants, anti-viral substances, enzyme inhibitors, hormones, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and antiadhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, angiogenic factors, anti-secretory factors, anticoagulants and/or antithrombotic agents, local anesthetics, prostaglandins, anti-depressants, anti-emetics, and imaging agents. In certain embodiments, the bioactive agent is a drug. Bioactive agents further include RNAs, such as siRNA, and osteoclast stimulating factors. In some embodiments, the bioactive agent may be a factor that stops, removes, or reduces the activity of bone growth inhibitors. In some embodiments, the bioactive agent is a growth factor, cytokine, extracellular matrix molecule or a fragment or derivative thereof, for example, a cell attachment sequence such as RGD.

In some embodiments, the pharmaceutical agent may include one or a plurality of therapeutic agents and/or pharmacological agents for release, including sustained release, to treat, for example, pain, inflammation, degeneration, or infection. The agent may be an analgesic agent including but are not limited to acetaminophen, a local anesthetic, such as for example, lidocaine, bupivicaine, ropivacaine, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, levorphanol, mepiridine, methadone, morphine, nalbuphine, opium, oxycodone, papaveretum, pentazocine, pethidine, phenoperidine, piritramide, dextropropoxyphene, remifentanil, sufentanil, tilidine, tramadol, codeine, dihydrocodeine, meptazinol, dezocine, eptazocine, flupirtine or a combination thereof.

The agent may be an anti-inflammatory agent including, but are not limited to, a statin, sulindac, sulfasalazine, naroxyn, diclofenac, indomethacin, ibuprofen, flurbiprofen, ketoprofen, aclofenac, aloxiprin, aproxen, aspirin, diflunisal, fenoprofen, mefenamic acid, naproxen, phenylbutazone, piroxicam, meloxicam, salicylamide, salicylic acid, desoxysulindac, tenoxicam, ketoralac, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, triflumidate, fenamates (mefenamic acid, meclofenamic acid), nabumetone, celecoxib, etodolac, nimesulide, apazone, gold, tepoxalin; dithiocarbamate, or a combination thereof. Anti-inflammatory agents also include other compounds such as steroids, such as for example, fluocinolone, cortisol, cortisone, hydrocortisone, fludrocortisone, prednisone, prednisolone, methylprednisolone, triamcinolone, betamethasone, dexamethasone, beclomethasone, fluticasone interleukin-1 receptor antagonists, thalidomide (a TNF-α release inhibitor), thalidomide analogues (which reduce TNF-α production by macrophages), bone morphogenetic protein (BMP) type 2 or BMP-4 (inhibitors of caspase 8, a TNF-α activator), quinapril (an inhibitor of angiotensin II, which upregulates TNF-α), interferons such as IL-11 (which modulate TNF-α receptor expression), and aurin-tricarboxylic acid (which inhibits TNF-α), guanidinoethyldisulfide, or a combination thereof. Exemplary anti-inflammatory agents include, for example, naproxen; diclofenac; celecoxib; sulindac; diflunisal; piroxicam; indomethacin; etodolac; meloxicam; ibuprofen; ketoprofen; r-flurbiprofen; mefenamic; nabumetone; tolmetin, and sodium salts of each of the foregoing; ketorolac bromethamine; ketorolac tromethamine; ketorolac acid; choline magnesium trisalicylate; rofecoxib; valdecoxib; lumiracoxib; etoricoxib; aspirin; salicylic acid and its sodium salt; salicylate esters of alpha, beta, gamma-tocopherols and tocotrienols (and all their d, l, and racemic isomers); methyl, ethyl, propyl, isopropyl, n-butyl, sec-butyl, t-butyl, esters of acetylsalicylic acid; tenoxicam; aceclofenac; nimesulide; nepafenac; amfenac; bromfenac; flufenamate; phenylbutazone, or a combination thereof.

An anti-inflammatory agent can be a steroid. Exemplary steroids include, for example, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, dexamethasone 21-acetate, dexamethasone 21-phosphate di-Na salt, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide or a combination thereof.

Anti-inflammatory agents also include those with anti-inflammatory properties, such as, for example, amitriptyline, carbamazepine, gabapentin, pregabalin, clonidine, or a combination thereof.

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.”

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims. Further, it should be understood that certain elements of the disclosed embodiments may be combined or exchanged with one another.

What is claimed is:

1. A bone growth composition, comprising:
demineralized bone fibers generated from a donor bone tissue;
cancellous chips generated from the donor bone tissue; and
a cryopreservative solution comprising glycerol in Lactated Ringer's, wherein a glycerol content of the cryopreservative solution is equal to or less than 25% by volume.

2. The bone growth composition of claim 1, wherein the cancellous chips are antimicrobial-treated with an antimicrobial agent.

3. The bone growth composition of claim 2, wherein the antimicrobial agent is not present or present in residual amounts of less than 1% by mass in the bone growth composition.

4. The bone growth composition of claim 1, wherein the demineralized bone fibers have an average length equal to or less than 5 mm and range in thickness between 0.05 to 0.5 mm.

5. The bone growth composition of claim 1, wherein the bone growth composition is enclosed in a container or a pouch.

6. The bone growth composition of claim 1, wherein the glycerol content of the cryopreservative solution is equal to or less than 10% by volume.

7. The bone growth composition of claim 1, wherein the cancellous chips and the fibers are present in about a 1:1 volume ratio.

8. The bone growth composition of claim 7, wherein there is less than 100% homogeneity of a cancellous portion of the donor bone tissue that is segmented and subsequently processed into the cancellous chips such that cancellous chips comprise at least 1% noncancellous material.

9. The bone growth composition of claim 7, wherein there is less than 100% homogeneity of a cortical or noncancellous portion of the donor bone tissue that is segmented and subsequently processed into the demineralized bone fibers, and wherein there is less than 100% homogeneity of the cortical portion such that the demineralized bone fibers comprise at least 1% noncortical material.

10. The bone growth composition of claim 1, further comprising one or both of corticocancellous chips and cortical chips in addition to the cancellous chips.

11. A frozen bone growth composition product, comprising:
a sealed flexible pouch enclosing a bone growth composition comprising:
demineralized bone fibers generated from a donor bone tissue;
cancellous chips generated from the donor bone tissue; and
a cryopreservative solution comprising glycerol in Lactated Ringer's, wherein a glycerol content of the cryopreservative solution is equal to or less than 20% by volume, wherein the demineralized bone fibers, the cancellous chips, and the cryopreservative solution are all frozen such that the bone growth composition is solid.

12. The frozen bone growth composition product of claim 11 wherein the cancellous chips and the fibers are present in about a 1:1 volume ratio.

13. The frozen bone growth composition product of claim 11, wherein the bone growth composition is frozen at a temperature from about −65° C. to about −80° C.

14. The frozen bone growth composition product of claim 11, wherein the bone growth composition is frozen at a temperature less than about −65° C. and wherein a viability of cells of the bone growth composition after thawing is more than 75-80%.

15. The frozen bone growth composition product of claim 11, wherein a total volume of the bone growth composition is 15 cubic centimeters or less.

16. The frozen bone growth composition product of claim 11, wherein the glycerol content of the cryopreservative solution is equal to or less than 10% by volume.

17. A thawed bone growth composition product, comprising:
a sealed flexible pouch enclosing a bone growth composition comprising:
demineralized bone fibers generated from a donor bone tissue;
cancellous chips generated from the donor bone tissue; and
a cryopreservative solution comprising glycerol in Lactated Ringer's, wherein a glycerol content of the cryopreservative solution is equal to or less than 20% by volume, wherein the demineralized bone fibers, the cancellous chips, and the cryopreservative solution are all thawed such that the bone growth composition is flowable within the sealed flexible pouch.

18. The thawed bone growth composition product of claim 17, wherein a total decantable volume of the cryopreservative solution after thawing is between about 0 mL to about 1 mL.

19. The thawed bone growth composition product of claim 17, wherein the sealed flexible pouch comprises a removable release liner covering a perforated wall.

20. The thawed bone growth composition product of claim 17, wherein the sealed flexible pouch is stored at 25° C. or higher after thawing.
